(19) United States
(12) Patent Application Publication
(10) Pub. No.: US 2023/0142973 A1
(43) Pub. Date: May 11, 2023

(54) WEARABLE DEVICES FOR TREATING AIR FOR INHALATION AND EXHALATION

(71) Applicant: SPIRA INNOVATION INC., Adstock (CA)

(72) Inventors: Yves GAMACHE, Adstock (CA); Andre LAMONTAGNE, Adstock (CA)

(21) Appl. No.: 17/914,955

(22) PCT Filed: Mar. 26, 2021
(86) PCT No.: PCT/CA2021/050398

§ 371 Date: Sep. 27, 2022

Related U.S. Application Data
(60) Provisional application No. 63/000,987, filed on Mar. 27, 2020.
(60) Provisional application No. 63/007,870, filed on Apr. 9, 2020.
(60) Provisional application No. 63/025,653, filed on May 15, 2020.
(60) Provisional application No. 63/113,496, filed on Nov. 13, 2020.

(51) Int. Cl. A62B 7/10; A61L 9/16; A62B 9/00; A62B 9/02; A62B 23/02; A62B 18/10; A62B 18/00; B01D 53/32; B01D 46/00

(52) U.S. Cl. CPC A62B 7/10; A61L 9/16; A62B 9/003; A62B 9/02; A62B 23/02; A62B 18/10; A62B 18/006; A62B 9/00; B01D 53/32; B01D 46/0028; A61L 2209/14; B01D 2259/818; B01D 2279/65

(57) ABSTRACT

Air to be inhaled by a user can be treated to remove contaminants by passing the air through a multistage treatment system. The multistage treatment system can include an inhalation treatment unit for treating air to be inhaled by the user, a mask wearable on the user's face, and an exhalation treatment unit for treating air exhaled by the user. The inhalation treatment unit and the exhalation treatment unit can each include various sub-units and assemblies for removing naturally occurring vapors and pathogens as well as compounds generated by operation of the treatment system.

### TECHNICAL FIELD

The technical field generally relates to techniques for treating air prior to inhalation by a user. In particular, the technical field relates to a system that includes a mask and a device for treating potentially contaminated air.

### BACKGROUND

Air can contain various undesirable components that can be inhaled by individuals. For example, various particulates, gases and pathogens can be present in air and can be harmful if inhaled. In addition, individuals who are infected with certain pathogens can exhale breath that includes undesirable pathogens, which can be a concern in terms of contamination or infection of others. There are a number of challenges with respect to treating air to remove contaminants prior to inhalation.

### SUMMARY

In accordance with an aspect, there is provided a device for treating contaminated air for inhalation by a user. The device comprises an inhalation treatment unit for treating air to be inhaled by the user, comprising:
a pressurized air intake section configured to receive the contaminated air, the pressurized air intake section comprising:
   a filter configured to separate particles from the contaminated air and produce a particle-reduced stream;
   an air pump to pressurize the particle-reduced stream; and
   a check-valve allowing forward flow of the pressurized particle-reduced stream while preventing backflow;
a thermal treatment section in fluid communication with the pressurized air intake section, the thermal treatment section comprising:
   a first contaminant removal unit configured to receive the particle-reduced stream and remove vapour contaminants selected from water, volatile organic compounds (VOCs), hydrocarbons, and CO₂;
   a heating unit configured to thermally treat the particle-reduced stream at a temperature to reduce a pathogen content thereof and produce a thermally treated stream;
   a cooling unit to reduce the temperature of the thermally treated stream; and
   a second contaminant removal unit configured to further remove vapour contaminants therefrom;
an ultraviolet (UV) treatment section in fluid communication with the thermal treatment section, the UV treatment section comprising:
   a UV chamber configured to receive the thermally treated stream; and
   a UV light source to emit UV radiation to contact the thermally treated stream within the UV chamber and remove pathogens therefrom, and produce a UV treated stream;
a plasma reactor section in fluid communication with the UV treatment section, the plasma reactor section comprising:
   a plasma chamber comprising a gas flow path allowing a flow of the UV treated stream to flow therethrough;
   a plasma generator configured to apply a plasma-generating field across the plasma chamber intersecting the flow of the UV treated stream to generate a plasma therefrom, thereby producing a plasma treated stream that includes plasma-generated compounds; and
   a third contaminant removal unit configured to receive the plasma treated stream and remove at least a portion of the plasma-generated compounds and produce treated air;
a buffer tank receiving the treated air;
a pressure regulator coupled to the buffer tank;
a humidifier coupled to the pressure regulator and receiving the treated air and producing a humidified treated air;
a feed inlet comprising an anti-return valve for supplying the humidified treated air; a mask coupled to the feed inlet for receiving the humidified treated air for inhalation by the user;
   an exhalation treatment unit coupled to the mask for treating the exhaled air from the user, the exhalation treatment unit comprising:
an outlet line coupled to the mask and configured to receive the exhaled air from the user, the outlet line comprising an anti-return valve;
an exhaust plasma reactor section in fluid communication with the outlet line, the exhaust plasma reactor section comprising:
   an exhaust plasma chamber comprising a gas flow path allowing a flow of the exhaled air therethrough; and
   an exhaust plasma generator configured to apply a plasma-generating field across the exhaust plasma chamber intersecting the flow of the exhaled air to generate a plasma therefrom, thereby producing treated exhaled air; and
an outlet coupled to the exhaust plasma reactor section for receiving the treated exhaled air and comprising an anti-return valve, for expelling the treated exhaled air to the atmosphere.

In some implementations, the thermal treatment section, the UV treatment section and the plasma reactor section are independently controlled.

In some implementations, the temperature of the thermal treatment is above about 250° C.

In some implementations, the temperature of the thermal treatment is between about 250° C. and 350° C.

In some implementations, the device further comprises a temperature sensor to monitor the temperature of the heating vessel, wherein the temperature sensor is operatively connected to a controller.

In some implementations, the cooling unit comprises an atmospheric heat sink.

In some implementations, the cooling unit reduces the temperature of the thermally treated stream between about 15° C. to 25° C.

In some implementations, the pathogens comprise at least one of viruses and bacteria.

In some implementations, the plasma-generating mechanism relies on a dielectric-barrier discharge.

In some implementations, the plasma-generating mechanism comprises an external electrode and an internal electrode, the external electrode and the internal electrode being operatively connected to a power supply providing an AC current.

In accordance with another aspect, there is provided a device for treating contaminated air for inhalation by a user. The device comprises:
an inhalation treatment unit for treating air to be inhaled by the user, comprising: a pressurized air intake section configured to receive and pressurize the contaminated air;
   a plasma reactor section in fluid communication with the pressurized air intake section, comprising:
      a plasma chamber comprising a gas flow path allowing a flow of the pressurized air to flow therethrough; and
      a plasma generator configured to apply a plasma-generating field across the plasma chamber intersecting the flow of the pressurized air to generate a plasma therefrom, thereby producing a plasma treated stream that includes plasma-generated compounds;
   a contaminant removal unit configured to receive the plasma treated stream and remove at least a portion of the plasma-generated compounds and produce treated air;
   a feed inlet for supplying the treated air;
a mask coupled to the feed inlet for receiving the treated air for inhalation by the user.

In some implementations, the pressurized air intake section comprises:
a filter configured to separate particles from the contaminated air;
an air pump to pressurize the contaminated air; and
a check-valve allowing forward flow of the pressurize the contaminated air while preventing backflow.

In some implementations, the device further comprises a buffer tank to receive the treated air.

In some implementations, the device further comprises a pressure regulator coupled to the buffer tank.

In some implementations, the device further comprises a humidifier coupled to the pressure regulator and receiving the treated air and producing a humidified treated air.

In some implementations, the feed inlet comprises an anti-return valve and is configured for supplying the humidified treated air to the mask.

In some implementations, the device further comprises an exhalation treatment unit coupled to the mask for treating the exhaled air from the user.

In some implementations, the exhalation treatment unit comprises:
an outlet line coupled to the mask and configured to receive the exhaled air from the user, the outlet line comprising an anti-return valve; and
an exhaust plasma reactor section in fluid communication with the outlet line.

In some implementations, the exhaust plasma reactor section comprises:
an exhaust plasma chamber comprising a gas flow path allowing a flow of the exhaled air therethrough; and
an exhaust plasma generator configured to apply a plasma-generating field across the exhaust plasma chamber intersecting the flow of the exhaled air to generate a plasma therefrom, thereby producing treated exhaled air.

In some implementations, the device further comprises an outlet coupled to the exhaust plasma reactor section for receiving the treated exhaled air and comprising an anti-return valve for expelling the treated exhaled air to the atmosphere.

In accordance with another aspect, there is provided a device for treating contaminated air for inhalation by a user. The device comprises:
an inhalation treatment unit for treating air to be inhaled by the user, comprising:
   a pressurized air intake section configured to receive the contaminated air, the pressurized air intake section comprising:
      an air pump to pressurize the contaminated air; and
      a check-valve allowing forward flow of the pressurized contaminated air while preventing backflow;
   at least one pathogen degradation unit coupled to the pressurized air intake section and configured for destroying pathogens to produce treated air and byproducts;
   a byproduct removal unit coupled to the at least one pathogen degradation unit and configured to remove at least a portion of the byproducts from the treated air;
   a feed inlet for supplying the treated air from the byproduct removal unit:
a mask coupled to the feed inlet for receiving the treated air for inhalation by the user.

In some implementations, the at least one pathogen degradation unit comprises at least one of a heating unit, a UV unit, and a plasma reactor unit.

In some implementations, the byproduct removal unit comprises an adsorbent.

In some implementations, the byproduct removal unit comprises at least one of a molecular sieve, a desiccant, and activated charcoal.

In accordance with another aspect, there is provided a device for treating contaminated air for inhalation by a user. The device comprises:
an inhalation treatment unit for treating air to be inhaled by the user, comprising:
   at least one treatment assembly configured for removing pathogens from the contaminated air to produce treated air; and
   a feed inlet for supplying the treated air from the treatment assembly to the user;
a mask coupled to the feed inlet for receiving the treated air for inhalation by the user; and
an exhalation treatment unit coupled to the mask for treating exhaled air from the user, the exhalation treatment unit comprising:
   an exhaust plasma reactor section in fluid communication with the outlet line, the exhaust plasma reactor section comprising:
      an exhaust plasma chamber comprising a gas flow path allowing a flow of the exhaled air therethrough; and
      an exhaust plasma generator configured to apply a plasma-generating field across the exhaust plasma chamber intersecting the flow of the exhaled air to generate a plasma therefrom, thereby producing treated exhaled air; and
   an outlet coupled to the exhaust plasma reactor section for expelling the treated exhaled air to the atmosphere.

In accordance with another aspect, there is provided a device for treating contaminated air for inhalation by a user. The device comprises:
an inhalation treatment unit for treating air to be inhaled by the user, in inhalation treatment unit comprising:
   a pressurized air intake section configured to receive the contaminated air and providing a feed pressure;
   at least one treatment assembly configured for removing pathogens from the contaminated air to produce treated air; and
   a feed inlet for supplying the treated air from the treatment assembly to the user;
a mask coupled to the inhalation treatment unit for receiving the treated air for inhalation by the user, the mask comprising a wall defining an inhalation chamber and having a surface for contacting a face of the user;
   wherein the feed pressure and the inhalation chamber are provided so as to pressurize the inhalation chamber to avoid infiltration of air from the atmosphere via gaps defined between the wall of the mask and the face of the user.

In accordance with another aspect, there is provided a device for treating contaminated air for inhalation by a user. The device comprises:
an inhalation treatment unit for treating air to be inhaled by the user;
a mask coupled to the inhalation treatment unit for receiving the treated air for inhalation by the user; and
an exhalation treatment unit coupled to the mask for treating exhaled air from the user;
wherein the device comprises one or more features as defined herein and/or as described and/or illustrated herein.

In accordance with another aspect, there is provided a method for treating contaminated air for inhalation by a user. The method comprises:
pre-treating air to be inhaled by the user, comprising:
   pressurizing the air to generate pressurized air; and
   subjecting the pressurized air to pathogen depletion to produce treated air depleted in pathogens;
feeding the treated air to the user; and
treating exhaled air from the user, comprising:
   subjecting the exhaled air to exhaust plasma treatment to produce a treated exhaled air; and
   expelling the treated exhaled air to atmosphere.

In some implementations, the pre-treating comprises filtering the air to remove particles prior to pressurizing and/or prior to the pathogen depletion.

In some implementations, the pre-treating comprises preventing backflow of the pressurized air from the pathogen depletion.

In some implementations, the pathogen depletion comprises one or more of thermal treatment, ultraviolet (UV) treatment, and plasma treatment.

In some implementations, the pathogen depletion comprises the thermal treatment, followed by the UV treatment, and then followed by the plasma treatment.

In some implementations, the pathogen depletion comprises the thermal treatment which comprises:
removing vapour contaminants from the air, the vapour contaminants being selected from water, volatile organic compounds (VOCs), hydrocarbons, and CO₂; and
heating the air to a temperature to degrade pathogens and thus reduce a pathogen content of the air and produce a thermally treated stream.

In some implementations, the thermal treatment further comprises:
cooling the thermally treated stream to produce cooled air; and
removing additional vapour contaminants from the cooled air.

In some implementations, the pathogen depletion comprises the UV treatment which comprises contacting the air with UV radiation to degrade potential pathogens therein and produce a UV treated stream.

In some implementations, the pathogen depletion comprises the plasma treatment which comprises applying a plasma-generating field across a flow of the air to generate a plasma therefrom, thereby degrading potential pathogens and producing a plasma treated stream that includes plasma-generated compounds.

In some implementations, the plasma treatment further comprises removing at least a portion of the plasma-generated compounds from the air to produce treated air.

In some implementations, the plasma-generated compounds include N₂O, NOx, and/or ozone.

In some implementations, the method comprises removing at least one precursor compound from the air prior to the plasma treatment, the at least one precursor compound being a compound that would be converted into undesirable contaminants by the plasma treatment.

In some implementations, the at least one precursor compound comprises CO₂ which is removed to avoid formation of CO as the undesirable contaminant.

In some implementations, the method further comprises accumulating the treated air prior to feeding the treated air to the user.

In some implementations, the treated air is accumulated in a buffer tank.

In some implementations, the method further comprises regulating pressure of the treated air to feed a pressure regulated air stream to the user.

In some implementations, the method further comprises humidifying the air prior to feeding the treated air to the user.

In some implementations, the method further comprises preventing backflow of the treated air.

In some implementations, feeding the treated air to the user comprises supplying the treated air into a mask worn by the user to enable inhalation.

In some implementations, the method further comprises preventing backflow of the exhaled air from the exhaust plasma treatment toward the user.

In some implementations, the method further comprises preventing backflow of the treated exhaled air back into the exhaust plasma treatment.

In accordance with another aspect, there is provided a method for treating contaminated air for inhalation by a user. The method comprises:
pre-treating air to be inhaled by the user, comprising:
   pressurizing the air to generate pressurized air; and
   subjecting the pressurized air to pathogen depletion to produce treated air depleted in pathogens;
feeding the treated air to the user; and
treating exhaled air from the user, comprising:
   subjecting the exhaled air to exhaust pathogen depletion to produce a treated exhaled air;
   preventing backflow of the exhaled air back toward the user; and
   expelling the treated exhaled air to atmosphere.

In some implementations, the method further comprises one or more features as defined herein and/or described herein and/or illustrated herein.

In some implementations, the pathogens include a virus.

In some implementations, the virus includes a SARS virus.

In some implementations, the SARS virus includes SARS-CoV-2.

In some implementations, the virus includes a MERS virus.

In accordance with another aspect, there is provided a device for treating contaminated air for inhalation by a user, the device comprising:
an inhalation treatment unit for treating air to be inhaled by the user, comprising:
   an air intake section configured to receive the contaminated air, the air intake section comprising:
      a filter configured to separate particles from the contaminated air and produce a particle-reduced stream; and
      an air pump to increase pressure of the contaminated air;
   a thermal treatment section in fluid communication with the air intake section, the thermal treatment section comprising:
      a heating unit configured to thermally treat the particle-reduced stream at a temperature sufficient to reduce a pathogen content thereof and produce a thermally treated stream, the heating unit comprising:
         a heating chamber;
         a first pathogen removal unit received within the heating chamber, the first pathogen removal unit being configured to receive the particle-reduced stream and provide a first porous region for vaporizing water and remove vapour contaminants therefrom;
         a second pathogen removal unit received within the heating chamber, the second pathogen removal unit being configured to provide a second porous region for vaporizing water and further remove vapour contaminants therefrom; and
         a cooling unit to reduce the temperature of the thermally treated stream;
   a contaminant removal unit configured to receive the thermally treated stream and remove contaminants selected from volatile organic compounds (VOCs), hydrocarbons, OH⁻, O₃, N₂O, CO, NOx and CO₂ and produce treated air;
   a pressure regulator coupled with the contaminant removal unit;
   a bacteria filter coupled to the pressure regulator;
   a humidifier coupled to the bacteria filter and configured to receive the treated air and producing a humidified treated air;
   a feed inlet comprising an anti-return valve for supplying the humidified treated air;
a mask coupled to the feed inlet for receiving the humidified treated air for inhalation by the user;
an exhalation treatment unit coupled to the mask for treating the exhaled air from the user, the exhalation treatment unit comprising:
   an outlet line coupled to the mask and configured to receive the exhaled air from the user, the outlet line comprising an anti-return valve;
   an exhaust plasma reactor section in fluid communication with the outlet line, the exhaust plasma reactor section comprising:
      an exhaust plasma chamber comprising a gas flow path allowing a flow of the exhaled air therethrough; and
      an exhaust plasma generator configured to apply a plasma-generating field across the exhaust plasma chamber intersecting the flow of the exhaled air to generate a plasma therefrom, thereby producing treated exhaled air; and
   an outlet coupled to the exhaust plasma reactor section for receiving the treated exhaled air and comprising an anti-return valve, for expelling the treated exhaled air to the atmosphere.

In some implementations, at least one of the first porous region and the second porous region is configured to retain pathogens therein.

In some implementations, the first porous region and the second porous region are configured to retain pathogens therein.

In some implementations, the second porous region has smaller pores than the first porous region.

In some implementations, the first porous region comprises a molecular sieve.

In some implementations, the second porous region comprises porous glass.

In some implementations, the first porous region comprises a heat exchanger and the second porous region comprises a molecular sieve.

In some implementations, the heating unit further comprises a third porous region.

In some implementations, the third porous region comprises porous glass.

In some implementations, the temperature of the thermal treatment is sufficient to produce superheated steam.

In accordance with another aspect, there is provided a device for treating contaminated air for inhalation by a user, the device comprising:
an inhalation treatment unit for treating air to be inhaled by the user, comprising:
   an air intake section configured to receive the contaminated air, the air intake section comprising:
      a filter configured to separate particles from the contaminated air and produce a particle-reduced stream; and
      an air pump to increase pressure of the contaminated air;
   a thermal treatment section in fluid communication with the air intake section, the thermal treatment section comprising:
      a heating unit configured to thermally treat the particle-reduced stream at a temperature sufficient to deactivate pathogens and produce a thermally treated stream, the heating unit comprising:
         a heating chamber;
         a first pathogen removal unit received within the heating chamber, the first pathogen removal unit being configured to receive the particle-reduced stream and provide a first porous region for vaporizing water and retain pathogens therein;
         a second pathogen removal unit received within the heating chamber, the second pathogen removal unit being configured to provide a second porous region for vaporizing water and further retain pathogens therein; and
         a cooling unit to reduce the temperature of the thermally treated stream;
   a contaminant removal unit configured to receive the thermally treated stream and remove contaminants selected from volatile organic compounds (VOCs), hydrocarbons, OH⁻, O₃, N₂O, CO, NOx and CO₂ and produce treated air;
   a pressure regulator coupled with the contaminant removal unit;
   a bacteria filter coupled to the pressure regulator;
   a humidifier coupled to the bacteria filter and configured to receive the treated air and producing a humidified treated air;
   a feed inlet comprising an anti-return valve for supplying the humidified treated air;
a mask coupled to the feed inlet for receiving the humidified treated air for inhalation by the user;
an exhalation treatment unit coupled to the mask for treating the exhaled air from the user, the exhalation treatment unit comprising:
   an outlet line coupled to the mask and configured to receive the exhaled air from the user, the outlet line comprising an anti-return valve;
   an exhaust plasma reactor section in fluid communication with the outlet line, the exhaust plasma reactor section comprising:
      an exhaust plasma chamber comprising a gas flow path allowing a flow of the exhaled air therethrough; and
      an exhaust plasma generator configured to apply a plasma-generating field across the exhaust plasma chamber intersecting the flow of the exhaled air to generate a plasma therefrom, thereby producing treated exhaled air; and
   an outlet coupled to the exhaust plasma reactor section for receiving the treated exhaled air and comprising an anti-return valve, for expelling the treated exhaled air to the atmosphere.

In some implementations, the second porous region has smaller pores than the first porous region.

In some implementations, the first porous region comprises a molecular sieve.

In some implementations, the second porous region comprises porous glass.

In some implementations, the temperature of the thermal treatment is sufficient to produce superheated steam.

In accordance with another aspect, there is provide a device for treating contaminated air for inhalation by a user, the device comprising:
an inhalation treatment unit for treating air to be inhaled by the user, comprising:
   an air intake section configured to receive the contaminated air;
   a thermal treatment section in fluid communication with the air intake section, the thermal treatment section comprising:
      a heating unit configured to thermally treat the contaminated air at a temperature sufficient to reduce a pathogen content thereof and produce a thermally treated stream, the heating unit comprising:
         a heating chamber;
         a pathogen removal unit received within the heating chamber, the pathogen removal unit being configured to receive the contaminated air and provide a porous region for vaporizing water present in the contaminated air and remove vapour contaminants therefrom;
   a contaminant removal unit configured to receive the thermally treated stream and remove byproducts from the thermal treatment section and produce treated air;
   a feed inlet comprising an anti-return valve for supplying the treated air;
a mask coupled to the feed inlet for receiving the treated air for inhalation by the user.

In some implementations, the pathogen removal unit is configured to increase a residence time of pathogens within the heating chamber.

In some implementations, the porous region is configured to retain pathogens therein.

In accordance with another aspect, there is provided a device for treating contaminated air for inhalation by a user, the device comprising:
a thermal treatment section comprising:
   a heating unit configured to thermally treat the contaminated air at a temperature sufficient to reduce a pathogen content thereof and produce treated air, the heating unit comprising:
      a heating chamber;
      a pathogen removal unit received within the heating chamber, the pathogen removal unit being configured to receive the contaminated air and provide a porous region for vaporizing water present in the contaminated air and remove vapour contaminants therefrom;
a feed inlet for supplying the treated air;
a mask coupled to the feed inlet for receiving the treated air for inhalation by the user.

In accordance with another aspect, there is provided a device for treating contaminated air for inhalation by a user, the device comprising:
a thermal treatment section comprising:
   a heating unit configured to thermally treat the contaminated air at a temperature sufficient to reduce a pathogen content thereof and produce treated air, the heating unit comprising:
      at least one pathogen removal unit configured to receive the contaminated air and provide a porous region for exposing pathogens to heat;
a feed inlet for supplying the treated air;
a mask coupled to the feed inlet for receiving the treated air for inhalation by the user.

In some implementations, the heating unit comprises a heating chamber, and the at least one pathogen removal unit is received within the heating chamber.

In some implementations, the at least one pathogen removal unit comprises a first pathogen removal unit comprising a heat exchanger.

In some implementations, the at least one pathogen removal unit comprises a second pathogen removal unit comprising a porous region configured to trap pathogens therein.

In accordance with another aspect, there is provided a method for treating contaminated air for inhalation by a user, the method comprising:
pre-treating air to be inhaled by the user, comprising:
   heating the contaminated air at a temperature sufficient to vaporize water and degrade pathogens to produce thermally treated air depleted in pathogens;
feeding the treated air to the user; and
treating exhaled air from the user, comprising:
   subjecting the exhaled air to exhaust pathogen depletion to produce a treated exhaled air;
   preventing backflow of the exhaled air back toward the user; and
   expelling the treated exhaled air to atmosphere.

In some implementations, heating the contaminated air comprises passing the contaminated air through a pathogen removal unit configured to provide a porous region for vaporizing water present in the contaminated air.

In some implementations, pre-treating air to be inhaled by the user further comprises filtering the air to remove particles prior to pathogen depletion.

In some implementations, pre-treating air to be inhaled by the user further comprises:
cooling the thermally treated air to produce cooled air; and
removing additional vapour contaminants from the cooled air.

In accordance with another aspect, there is provided a device for treating contaminated air for inhalation by a user, the device comprising:
an inhalation treatment unit for treating air to be inhaled by the user, comprising:
   an air intake section configured to receive the contaminated air, the air intake section comprising:
      a filter configured to separate particles from the contaminated air and produce a particle-reduced stream; and
      an air pump to increase pressure of the contaminated air;
   a thermal treatment section in fluid communication with the air intake section, the thermal treatment section comprising:
      a heating unit configured to thermally treat the particle-reduced stream at a temperature sufficient to deactivate pathogens contained therein and produce a thermally treated stream, the heating unit comprising:
         a heating chamber;
         a heating element configured to provide heat to the heating chamber;
         a pathogen removal unit received within the heating chamber, the pathogen removal unit being configured to provide a porous region to retain the pathogens therein; and
         a cooling unit to reduce the temperature of the thermally treated stream;
   a contaminant removal unit configured to receive the thermally treated stream and remove contaminants selected from volatile organic compounds (VOCs), hydrocarbons, OH⁻, O₃, N₂O, CO, NOx and CO₂ and produce treated air;
   a flow control valve coupled with the contaminant removal unit;
   a bacteria filter coupled to the flow control valve;
   a feed inlet in fluid communication with the flow control valve, the feed inlet comprising an anti-return valve for supplying the treated air;
a mask coupled to the feed inlet for receiving the humidified treated air for inhalation by the user;
an exhalation treatment unit coupled to the mask for treating the exhaled air from the user, the exhalation treatment unit comprising:
   an outlet line coupled to the mask and configured to receive the exhaled air from the user, the outlet line comprising an anti-return valve;
   an exhaust plasma reactor section in fluid communication with the outlet line, the exhaust plasma reactor section comprising:
      an exhaust plasma chamber comprising a gas flow path allowing a flow of the exhaled air therethrough; and
      an exhaust plasma generator configured to apply a plasma-generating field across the exhaust plasma chamber intersecting the flow of the exhaled air to generate a plasma therefrom, thereby producing treated exhaled air; and
   an outlet coupled to the exhaust plasma reactor section for receiving the treated exhaled air and comprising an anti-return valve, for expelling the treated exhaled air to the atmosphere.

In accordance with another aspect, there is provided a device for treating contaminated air for inhalation by a user, the device comprising:
an inhalation treatment unit for treating air to be inhaled by the user, comprising:
   an air intake section configured to receive the contaminated air, the air intake section comprising:
      a filter configured to separate particles from the contaminated air and produce a particle-reduced stream; and
   a thermal treatment section in fluid communication with the air intake section, the thermal treatment section comprising:
      a heating unit configured to thermally treat the particle-reduced stream at a temperature sufficient to deactivate pathogens contained therein and produce a thermally treated stream, the heating unit comprising:
         a heating chamber;
         a heating element configured to provide heat to the heating chamber;
         a pathogen removal unit received within the heating chamber, the pathogen removal unit being configured to provide a porous region to retain the pathogens therein; and
   a contaminant removal unit configured to receive the thermally treated stream and remove contaminants therefrom and produce treated air;
   a flow control valve coupled with the contaminant removal unit;
   an air pump to suction in the contaminated air into the air intake section and downstream sub-units; and
   a feed inlet in fluid communication with the air pump, the feed inlet comprising an anti-return valve for supplying the treated air; and
a mask coupled to the feed inlet for receiving the treated air for inhalation by the user.

In some implementations, the porous region has a pore size between about 1 nm and 10 nm.

In some implementations, the porous region has a pore size of less than about 1 nm.

In some implementations, the porous region comprises a metal mesh.

In some implementations, the metal mesh comprises sintered metal fibers.

In some implementations, the metal mesh comprises multiple layers of sintered metal fibers to form a multi-layer metal mesh.

In some implementations, the sintered metal fibers are configured to lay substantially uniformly to form a three-dimensional non-woven structure.

In some implementations, the three-dimensional non-woven structure is sintered at contact points.

In some implementations, the pore size of at least one layer of the multi-layer metal mesh is different than the pore size of the remaining layers.

In some implementations, the heating unit further comprises a heat exchanger configured to be received within the heating chamber.

In some implementations, the heat exchanger is provided upstream of the porous region.

In some implementations, the heat exchanger is configured to provide an additional porous region.

In some implementations, the additional porous region has larger pores than the porous region.

In some implementations, the heat exchanger comprises a metal wool.

In some implementations, the metal wool comprises at least one of a stainless steel wool and a copper wool.

In some implementations, the heating element comprises a heater cartridge.

In some implementations, the heating element is configured to be surrounded by the heat exchanger.

In some implementations, the pathogen removal unit is configured to increase a residence time of the pathogens within the heating chamber.

In some implementations, the device further comprises a temperature sensor to monitor the temperature within the heating chamber.

In some implementations, the device further comprises a controller operatively connected to the temperature sensor and to the heating element, the controller being configured to adjust the temperature within the thermal unit in response to a measured temperature value provided by the temperature sensor.

In some implementations, the controller is configured to adjust the temperature within the thermal unit according to a heating cycle.

In some implementations, the heating cycle comprises a temperature sequence comprising a low temperature setpoint and a high temperature setpoint.

In accordance with another aspect, there is provided a device for treating contaminated air for inhalation by a user, the device comprising:
an inhalation treatment unit for treating air to be inhaled by the user, comprising:
   an air intake section configured to receive the contaminated air;
   a thermal treatment section in fluid communication with the air intake section, the thermal treatment section comprising:
      a heating unit configured to thermally treat the contaminated air at a temperature sufficient to deactivate pathogens contained therein and produce a thermally treated stream, the heating unit comprising:
         a heating chamber;
         a heating element configured to provide heat to the heating chamber;
         a pathogen removal unit received within the heating chamber, the pathogen removal unit being configured to provide a porous region to retain the pathogens therein;
   a contaminant removal unit configured to receive the thermally treated stream and remove byproducts from the thermal treatment section and produce treated air;
   a feed inlet comprising an anti-return valve for supplying the treated air;
a mask coupled to the feed inlet for receiving the treated air for inhalation by the user.

In some implementations, the porous region comprises a metal mesh.

In some implementations, the porous region has a pore size between about 1 nm and 10 nm.

In some implementations, the porous region has a pore size of less than about 1 nm.

In some implementations, the pathogen removal unit is configured to increase a residence time of pathogens within the heating chamber.

In some implementations, the air intake section comprises a filter configured to separate particles from the contaminated air, and an air pump to pressurize the contaminated air.

In some implementations, the device further comprises an exhalation treatment unit coupled to the mask for treating the exhaled air from the user.

In some implementations, the exhalation treatment unit comprises:
an outlet line coupled to the mask and configured to receive the exhaled air from the user, the outlet line comprising an anti-return valve; and
an exhaust plasma reactor section in fluid communication with the outlet line.

In some implementations, the exhaust plasma reactor section comprises:
an exhaust plasma chamber comprising a gas flow path allowing a flow of the exhaled air therethrough; and
an exhaust plasma generator configured to apply a plasma-generating field across the exhaust plasma chamber intersecting the flow of the exhaled air to generate a plasma therefrom, thereby producing treated exhaled air.

In some implementations, the device further comprises an outlet coupled to the exhaust plasma reactor section for receiving the treated exhaled air and comprising an anti-return valve for expelling the treated exhaled air to the atmosphere.

In accordance with another aspect, there is provided a device for treating contaminated air for inhalation by a user, the device comprising:
a thermal treatment section comprising:
   a heating unit configured to thermally treat the contaminated air at a temperature sufficient to deactivate pathogens contained therein and produce treated air, the heating unit comprising:
      a heating chamber;
      a heating element configured to provide heat to the heating chamber;
      a pathogen removal unit received within the heating chamber, the pathogen removal unit being configured to receive the contaminated air and provide a porous region to retain the pathogens therein and expose the retained pathogens to heat;
   a temperature sensor to monitor the temperature within the heating chamber;
   a controller operatively connected to the temperature sensor and to the heating element to control the temperature within the heating chamber according to a heating cycle;
a feed inlet for supplying the treated air;
a mask coupled to the feed inlet for receiving the treated air for inhalation by the user.

In accordance with another aspect, there is provided a device for treating contaminated air for inhalation by a user, the device comprising:
a thermal treatment section comprising:
   a heating unit configured to thermally treat the contaminated air at a temperature sufficient to deactivate pathogens contained therein and produce treated air, the heating unit comprising:
      at least one pathogen removal unit configured to receive the contaminated air and provide a porous region to retain the pathogens therein and expose the retained pathogens to heat;
   a feed inlet for supplying the treated air;
a mask coupled to the feed inlet for receiving the treated air for inhalation by the user.

In some implementations, the heating unit comprises a heating chamber, and the at least one pathogen removal unit is received within the heating chamber.

In some implementations, the at least one pathogen removal unit comprises a first pathogen removal unit comprising a heat exchanger.

In some implementations, the at least one pathogen removal unit comprises a second pathogen removal unit comprising a porous region configured to trap pathogens therein.

In accordance with another aspect, there is provided a method for treating contaminated air for inhalation by a user, the method comprising:
pre-treating air to be inhaled by the user, comprising:
   heating the contaminated air at a temperature sufficient to deactivate pathogens to produce thermally treated air depleted in pathogens;
feeding the treated air to the user; and
treating exhaled air from the user, comprising:
   subjecting the exhaled air to exhaust pathogen depletion to produce a treated exhaled air;
   preventing backflow of the exhaled air back toward the user; and
   expelling the treated exhaled air to atmosphere.

In some implementations, heating the contaminated air comprises passing the contaminated air through a pathogen removal unit configured to provide a porous region for retaining the pathogens therein.

In some implementations, pre-treating air to be inhaled by the user further comprises filtering the air to remove particles prior to pathogen depletion.

In some implementations, pre-treating air to be inhaled by the user further comprises:
cooling the thermally treated air to produce cooled air; and
removing additional vapour contaminants from the cooled air.

In accordance with another aspect, there is provided a method for treating contaminated air for inhalation by a user, the method comprising:
thermally treating the contaminated air, comprising:
   receiving the contaminated air comprising pathogens within a porous region of a heating chamber of a heating unit, the porous region being configured to retain the pathogens therein;
   subjecting the pathogens retained in the porous region to a heating cycle comprising at least a first phase and a second phase, comprising:
      supplying heat to the heating chamber via a heating element located in proximity of the heating chamber, wherein in the first phase, the supplying of the heat is performed according to a first temperature setpoint, and wherein in the second phase, the supplying of the heat is performed according to a second temperature setpoint that is different from the first temperature setpoint; and
feeding the treated air to the user.

In some implementations, the first temperature setpoint is lower than the second temperature setpoint.

In some implementations, at least one of the first temperature setpoint and the second temperature setpoint is set at a temperature sufficiently high to deactivate the pathogens.

In accordance with another aspect, there is provided a device for treating contaminated air for inhalation by a user, the device comprising:
an inhalation treatment unit for treating air to be inhaled by the user, comprising:
   an air intake section configured to receive the contaminated air, the air intake section comprising:
      an air pump to increase pressure of the contaminated air;
   a filtering unit in fluid communication with the air intake section, the filtering unit comprising:
      a filter comprising a porous material configured to retain pathogens therein to produce treated air, the filter being configured to be subjected to a filtration stage and a cleaning stage;
   a feed inlet comprising an anti-return valve for supplying the treated air; and
a mask coupled to the feed inlet for receiving the treated air for inhalation by the user.

In some implementations, the filter is configured to be subjected to the filtration stage and the cleaning stage substantially simultaneously.

In some implementations, the filter is configured to be subjected to the filtration stage and the cleaning stage sequentially.

In some implementations, the filter is removable from the filtering unit and the inhalation treatment.

In some implementations, the filter is removable from the filtering unit and the inhalation treatment to be subjected to the cleaning stage.

In some implementations, the filter is configured to be reusable following the cleaning stage.

In some implementations, the filter is configured to be subjected to a thermal treatment outside of the inhalation treatment unit during the cleaning stage.

In some implementations, the porous material is configured to sustain temperatures above 50° C. during the thermal treatment.

In some implementations, the filter is configured to be subjected to a plasma treatment during the cleaning stage to deactivate pathogens trapped in the filter.

In some implementations, the filtering unit further comprises:
a casing defining a cartridge-receiving section; and
a cartridge comprising a filter-retaining frame for holding the filter in position, the cartridge being receivable into the cartridge-receiving section.

In some implementations, the cartridge is removable from the cartridge-receiving section to remove the filter from the filtering unit and the inhalation treatment.

In some implementations, the filter-retaining frame is configured to be slidably insertable into the cartridge-receiving section.

In some implementations, the filter-retaining frame is configured to be received into the cartridge-receiving section via a snap-on mechanism.

In some implementations, the cartridge is receivable into the cartridge-receiving section to provide an air-tight seal around the cartridge.

In some implementations, the filter has a thickness ranging from about 5 mm to about 1 cm.

In some implementations, the porous material has a pore size between about 1 nm and about 10 nm.

In some implementations, the porous material has a pore size of between about 1 nm and 5 nm.

In some implementations, the porous material is made of a metal.

In some implementations, the porous material is made of stainless steel.

The device of claim 143, wherein the porous material is made of copper.

In some implementations, the porous material comprises sintered metal fibers.

In some implementations, the porous material comprises multiple layers of sintered metal fibers to form a multi-layer porous material.

In some implementations, the filter is configured such that an electrical field can be applied therethrough during the cleaning stage to deactivate pathogens trapped in the filter.

In some implementations, the porous material is configured to retain pathogens having a mass median aerodynamic diameter of about 300 nm or less.

In some implementations, the porous material is configured to retain pathogens having a mass median aerodynamic diameter of between about 300 nm and about 100 nm.

In some implementations, the porous material is configured to retain pathogens having a mass median aerodynamic diameter of between about 100 nm and about 50 nm.

In some implementations, the porous material is configured to retain pathogens having a mass median aerodynamic diameter of between about 50 nm and about 10 nm.

In some implementations, the porous material is configured to retain pathogens having a mass median aerodynamic diameter of between 10 nm and 1 nm.

In some implementations, the porous material is configured to retain pathogens having a mass median aerodynamic diameter of less than about 5 nm.

In some implementations, the porous material is configured to retain pathogens having a mass median aerodynamic diameter of less than about 2 nm.

In some implementations, the porous material is configured to retain therein 99% of pathogens having a diameter larger than 1.5 nm.

In some implementations, the device further comprises an exhalation treatment unit in fluid communication with the mask for treating the exhaled air from the user.

In some implementations, the exhalation treatment unit comprises an outlet line coupled to the mask and configured to receive the exhaled air from the user, the outlet line comprising an anti-return valve for expelling the treated exhaled air to the atmosphere.

In some implementations, the exhalation treatment unit comprises at least one of an exhaust plasma reactor and an additional filtering unit as defined in any one of claims 126 to 156.

In some implementations, the device comprises one or more features as defined in any above claim and/or as described and/or illustrated herein.

In accordance with another aspect, there is provided a device for treating contaminated air for inhalation by a user, the device comprising:
a filtering unit for treating air to be inhaled by the user, the filtering unit comprising:
   a filter comprising a porous material configured to retain pathogens therein to produce treated air, the filter being configured to be subjected to a filtration stage and a cleaning stage;
a mask comprising:
   a filtering unit-receiving opening configured to receive the filtering unit therein; and
   an outer surface and an inner surface, the inner surface and the user's face defining an inhalation chamber for receiving the treated air for inhalation by the user.

In some implementations, the filter is configured to be subjected to the filtration stage and the cleaning stage substantially simultaneously.

In some implementations, the filter is configured to be subjected to the filtration stage and the cleaning stage sequentially.

In some implementations, the filter is removable from the filtering unit.

In some implementations, the filter is removable from the filtering unit to be subjected to the cleaning stage.

In some implementations, the filter is configured to be reusable following the cleaning stage.

In some implementations, the filter is configured to be subjected to a thermal treatment outside of the inhalation treatment unit during the cleaning stage.

In some implementations, the porous material is configured to sustain temperatures above 50° C. during the thermal treatment.

In some implementations, the filter is configured to be subjected to a plasma treatment during the cleaning stage to deactivate pathogens trapped in the filter.

In some implementations, the filtering unit further comprises:
a casing defining a cartridge-receiving section; and
a cartridge comprising a filter-retaining frame for holding the filter in position, the cartridge being receivable into the cartridge-receiving section.

In some implementations, the cartridge is removable from the cartridge-receiving section to remove the filter from the filtering unit.

In some implementations, the filter-retaining frame is configured to be slidably insertable into the cartridge-receiving section of the casing.

In some implementations, the filter-retaining frame is configured to be received into the cartridge-receiving section via a snap-on mechanism.

In some implementations, the cartridge is receivable into the cartridge-receiving section to provide an air-tight seal around the cartridge.

In some implementations, the filter has a thickness ranging from about 5 mm to about 1 cm.

In some implementations, the porous material has a pore size between about 1 nm and about 10 nm.

In some implementations, the porous material has a pore size of between about 1 nm and 5 nm.

In some implementations, the porous material is made of a metal.

In some implementations, the porous material is made of stainless steel.

In some implementations, the porous material is made of copper.

In some implementations, the porous material comprises sintered metal fibers.

In some implementations, the porous material comprises multiple layers of sintered metal fibers to form a multi-layer porous material.

In some implementations, the filter is configured such that an electrical field can be applied therethrough during the cleaning stage to deactivate pathogens trapped in the filter.

In some implementations, the porous material is configured to retain pathogens having a mass median aerodynamic diameter of about 300 nm or less.

In some implementations, the porous material is configured to retain pathogens having a mass median aerodynamic diameter of between about 300 nm and about 100 nm.

In some implementations, the porous material is configured to retain pathogens having a mass median aerodynamic diameter of between about 100 nm and about 50 nm.

In some implementations, the porous material is configured to retain pathogens having a mass median aerodynamic diameter of between about 50 nm and about 10 nm.

In some implementations, the porous material is configured to retain pathogens having a mass median aerodynamic diameter of between 10 nm and 1 nm.

In some implementations, the porous material is configured to retain pathogens having a mass median aerodynamic diameter of less than about 5 nm.

In some implementations, the porous material is configured to retain pathogens having a mass median aerodynamic diameter of less than about 2 nm.

In some implementations, the porous material is configured to retain therein 99% of pathogens having a diameter larger than 1.5 nm.

In some implementations, the device comprises one or more features as defined in any above claim and/or as described and/or illustrated herein.

In accordance with another aspect, there is provided a device for treating contaminated air, the device comprising:
at least one of an inhalation treatment unit coupled to a mask wearable on a user's face for treating air to be inhaled by the user and an exhalation treatment unit coupled to the mask wearable for treating exhaled air from the user, the at least one of the inhalation treatment unit and the exhalation treatment unit comprising:
   a filtering unit configured to receive the contaminated air, the filtering unit comprising:
      a filter comprising a porous material configured to retain pathogens therein to produce treated air and being configured to be cycled between a filtration stage and a cleaning stage.

In some implementations, the device comprises one or more features as defined in any above claim and/or as described and/or illustrated herein.

In accordance with another aspect, there is provided a device for treating contaminated air for inhalation by a user, the device comprising:
a filtering unit for treating air to be inhaled by the user, the filtering unit comprising:
   a filter comprising a metallic porous material configured to retain pathogens therein to produce treated air, the filter being configured to be subjected to a filtering cycle and a cleaning cycle;
a mask in fluid communication with the filtering unit for receiving the treated air for inhalation by the user.

In some implementations, the filter is configured to be subjected to the filtration stage and the cleaning stage substantially simultaneously.

In some implementations, the filter is configured to be subjected to the filtration stage and the cleaning stage sequentially.

In some implementations, the filter is removable from the filtering unit to be subjected to the cleaning stage.

In some implementations, the filter is configured to be reusable following the cleaning stage.

In some implementations, the filtering unit further comprises:
a casing defining a cartridge-receiving section;
a cartridge comprising a filter-retaining frame for holding the filter in position, the cartridge being receivable into the cartridge-receiving section; and
an electrically insulated gasket to electrically insulate the filter.

In some implementations, the cartridge is removable from the cartridge-receiving section to remove the filter from the filtering unit and the inhalation treatment.

In some implementations, the filter-retaining frame is configured to be slidably insertable into the cartridge-receiving section.

In some implementations, the filter-retaining frame is configured to be received into the cartridge-receiving section via a snap-on mechanism.

In some implementations, the cartridge is receivable into the cartridge-receiving section to provide an air-tight seal around the cartridge.

In some implementations, the metallic porous material is made of stainless steel.

In some implementations, the metallic porous material is made of copper.

In some implementations, the metallic porous material comprises sintered metal fibers.

In some implementations, the porous material comprises multiple layers of sintered metal fibers to form a multi-layer porous material.

In some implementations, the metallic porous material forms a first electrode.

In some implementations, the cartridge forms a second electrode.

In some implementations, the filter is configured such that an electrical field can be applied the first electrode and the second electrode during the cleaning stage to deactivate pathogens trapped in the filter.

In some implementations, the filter is connected to a DC high voltage source or an AC voltage source.

In some implementations, the device comprises one or more features as defined in any above claim and/or as described and/or illustrated herein.

In accordance with another aspect, there is provided a device for treating contaminated air for inhalation by a user, the device comprising:
a filtering unit for treating air to be inhaled by the user, the filtering unit comprising:
   a filter comprising a porous material configured to retain pathogens therein to produce treated air, the filter being configured to be subjected to a filtering cycle and a cleaning cycle;
   a plasma generator configured to apply a plasma-generating field across the filter to generate a plasma therefrom during the cleaning stage; and
a mask in fluid communication with the filtering unit for receiving the treated air for inhalation by the user.

In some implementations, the filter is configured to be subjected to the filtration stage and the cleaning stage substantially simultaneously.

In some implementations, the filtration unit further comprises a contaminant removal unit configured to receive a plasma treated stream and remove at least a portion of plasma-generated compounds produced during the cleaning stage.

In some implementations, the filter is configured to be subjected to the filtration stage and the cleaning stage sequentially.

In some implementations, the filter is configured to be reusable following the cleaning stage.

In some implementations, the device comprises one or more features as defined in any above claim and/or as described and/or illustrated herein.

In accordance with another aspect, there is provided a method for treating contaminated air for inhalation by a user, the method comprising:
subjecting the contaminated air to a filtering stage to produce treated air depleted in pathogens, comprising:
   passing the contaminated air in an inflow direction through a filter of a filtering unit that is in fluid communication with a mask wearable on the user's face, the filter comprising a porous material configured to retain pathogens therein to produce treated air to be inhaled by the user; and
subjecting the filter to a cleaning stage to deactivate pathogens retained in the filter.

In some implementations, the method further comprises removing the filter from the filtering unit following a given usage duration, subjecting the filter to the cleaning stage and placing the filter back into the filtering unit for a subsequent filtering cycle.

In some implementations, the cleaning stage is performed while the mask is in use.

In some implementations, the cleaning stage is performed while the mask is removed from the user's face.

In some implementations, the porous material is a metallic porous material, and the cleaning stage comprises applying an electrical field through the filter to deactivate the pathogens retained in the filter.

In some implementations, the cleaning stage comprises subjecting the filter to a plasma treatment.

In some implementations, the cleaning stage comprises a wet thermal treatment, the wet thermal treatment comprising:
submerging the filter into an aqueous medium;
heating the aqueous medium at a temperature and for a duration sufficient to at least deactivate pathogens.

In some implementations, the wet thermal treatment is performed at a temperature of at least 100° C.

In some implementations, the wet thermal treatment is performed for a duration of at least 1 minute.

In some implementations, the wet thermal treatment is performed for a duration ranging from between about 1 minute and 5 minutes.

In some implementations, the cleaning stage comprises a dry thermal treatment, the dry thermal treatment comprising:
heating the filter at a temperature and for a duration sufficient to at least deactivate pathogens.

In some implementations, the dry thermal treatment is performed at a temperature of at least 65° C.

In some implementations, the dry thermal treatment is performed for a duration ranging between about 5 minutes to about 30 minutes.

In some implementations, the filtering unit is integrated into the mask.

In some implementations, the method further comprises treating exhaled air from the user by passing the exhaled air through the filter in an outflow direction, reversed from the inflow direction.

In some implementations, the filtering unit is provided as a separate component from the mask.

In some implementations, the method further comprises:
treating exhaled air from the user, comprising:
   subjecting the exhaled air to exhaust pathogen depletion to produce a treated exhaled air;
   preventing backflow of the exhaled air back toward the user; and
   expelling the treated exhaled air to atmosphere.

In some implementations, subjecting the exhaled air to exhaust pathogen depletion comprises passing the exhaled air through an additional filtering unit in fluid communication with the mask to produce the treated exhaled air.

### BRIEF DESCRIPTION OF THE DRAWINGS

FIG. 1 is a schematic representation of an implementation of a multistage treatment system for treating air to be inhaled and exhaled air, the multistage treatment system including an inhalation treatment unit that includes an air intake section, a thermal treatment section, a UV treatment section, a plasma reactor section, and a pre-inhalation treatment section, and an exhaust plasma reactor section.

FIG. 2 is a front view of a coaxial plasma reactor for use as part of an exhaust plasma reactor section of a multistage treatment system.

FIG. 3 is a side view of the coaxial plasma reactor of FIG. 2.

FIG. 4A is a side view of a coaxial plasma reactor with a solid conductive inner electrode.

FIG. 4B is a side view of a coaxial plasma reactor with solid conductive inner electrode and dialectic beads.

FIG. 4C is a side view of a coaxial plasma reactor with electrically conductive beads.

FIG. 5 is an example of a plasma reactor section and a pre-inhalation treatment section of a multistage treatment system.

FIG. 6 is a schematic representation of a multistage treatment system connected to a mask worn on a user's face.

FIG. 7 is a schematic representation of an implementation of a multistage treatment system for treating air to be inhaled and exhaled air, the multistage treatment system including an inhalation treatment unit that includes an air intake section, a thermal treatment section and a pre-inhalation treatment section, and an exhaust plasma reactor section.

FIG. 8 is a schematic representation of a sequence of treatment of contaminated air.

FIG. 9 is a schematic representation of a superheating process to free pathogens from water droplets.

FIG. 10 is a schematic representation of pathogens being trapped in a porous material.

FIG. 11 is a side view of a heating unit for use as part of a thermal treatment section of a multistage treatment system.

FIG. 12A is a front view of a heat dissipator that includes a porous material.

FIG. 12B is a front view of a middle section of the heat dissipator of FIG. 12A.

FIG. 12C is a top view of the heat dissipator of FIG. 12A.

FIG. 13 is a side view of a heating unit and associated graph showing air molecule velocity and pathogen velocity as a function of traveled distance in the heating unit.

FIG. 14 is a side view of a plasma reactor with solid conductive inner electrode and dielectric porous material, and associated showing air molecule velocity and pathogen velocity as a function of traveled distance in the heating unit.

FIG. 15 is a side view of the plasma reactor of FIG. 14, showing enlarged portions of various sections of the plasma reactor, and associated showing air molecule velocity and pathogen velocity as a function of traveled distance in the heating unit.

FIG. 16 is a schematic representation of a mask worn on a user's face, the mask being coupled to a multistage treatment system, showing treated air exiting the mask to prevent ambient air leak ingress.

FIG. 17A is another schematic representation of an implementation of a multistage treatment system for treating air to be inhaled and exhaled air, the multistage treatment system including an inhalation treatment unit that includes an air intake section, a thermal treatment section and a pre-inhalation treatment section, and an exhaust plasma reactor section.

FIG. 17B is another schematic representation of an implementation of a multistage treatment system for treating air to be inhaled and exhaled air, the multistage treatment system including an inhalation treatment unit that includes an air intake section, a thermal treatment section and a pre-inhalation treatment section, and an exhaust plasma reactor section.

FIG. 18 is another schematic representation of pathogens being trapped in a porous material.

FIG. 19 is side view of a heating unit that includes a heat dissipator, a heater, a temperature sensor and a porous material.

FIG. 20 illustrates a first graph showing variations in temperature as a function of time and a second graph showing variations of heating power as a function of time in connection with power usage of a thermal unit of a multistage treatment system.

FIG. 21 is a perspective view of a filtration unit that includes a casing defining a cartridge-receiving section and an associated cartridge.

FIG. 22 is a perspective view of the cartridge shown in FIG. 21.

FIGS. 23A-23B illustrate examples of a mask having a filtration unit integrated into the mask.

FIG. 24 illustrates an implementation of a filtration unit that includes four filters provided in a side-by-side relationship, the filtration unit being portable by a user.

FIG. 25 illustrates a first graph showing variations in pressure in an inhalation chamber of a mask as a function of time, a second graph showing variations in blower speed in the inhalation chamber as a function of time, and a third graph showing variations in blower power in the inhalation chamber as a function of time.

### DETAILED DESCRIPTION

Air can be subjected to treatments for removing contaminants that may be harmful if inhaled.

For instance, ambient air to be inhaled by a user can be treated to remove contaminants by subjecting the contaminated air to a multistage treatment process that can be implemented by a multistage treatment system. The multistage treatment system can include an inhalation treatment unit for treating air to be inhaled by the user, a mask wearable on the user's face, and an exhalation treatment unit for treating exhaled air. The inhalation treatment unit and the exhalation treatment unit can include various sub-units and assemblies, some examples of which are described below.

For example, the inhalation treatment unit can include at least one contaminant removal unit, and optionally a byproducts removal unit, that can be configured to remove selected contaminants, such as particles, volatile organic compounds (VOCs), hydrocarbons, CO₂, NOx, and ozone, for instance. The contaminant removal unit, of which there can be several, can be configured to remove compounds that naturally occur in the air, but are undesirable to be present in a subsequent stage of the inhalation treatment unit (e.g., CO₂ in some implementations) and/or are undesirable for inhalation by the user (e.g., VOCs, particles). The byproduct removal unit can be configured to remove compounds that do not necessarily occur in notable quantities in natural air, but are generated by one or more of the stages of the inhalation treatment unit and are thus ideally removed prior to inhalation by the user (e.g., NOx, ozone).

The inhalation treatment unit can also include one or more pathogen degradation units. Examples of pathogen degradation units include a thermal treatment section, an ultraviolet (UV) treatment section, and a plasma reactor section. The inhalation treatment unit is coupled to the mask that the user wears on her face such that the user can inhale the treated air. The inhalation treatment unit is configured to define at least one fluid passage that fluidly connects the atmosphere to the interior of the mask so that the air can pass through the various treatment units prior to inhalation of the treated air. The fluid passage can include certain tubes or conduits, as well as the chambers of the various units that are present in the inhalation treatment unit. In some implementations, all three of the pathogen degradation units can be included in series in the inhalation treatment unit, preferably in the following order: thermal treatment section, ultraviolet (UV) treatment section, and then plasma reactor section. However, it is noted that various different arrangements and combination of the pathogen degradation units can be provided to achieve redundancy and efficiencies in terms of the destruction of pathogens, such as bacteria and viruses.

The mask is also coupled to the exhalation treatment unit. The exhalation treatment unit enables treatment of the exhaled air from the user. Treatment of the exhaled air can be useful in situations when the air exhaled by the user contains potentially pathogens, such as viruses or bacteria, and release of these pathogens in ambient air may be harmful to other individuals that could be exposed to such contaminated air. The exhalation treatment unit can include one or more exhaust pathogen degradation units, such as an exhaust plasma reactor section. The exhalation treatment unit is also configured to define a fluid passage that fluidly connects the interior of the mask to the atmosphere so that the treated exhaled air can pass through the exhaust pathogen degradation unit prior to being released into the atmosphere. The fluid passage can also include certain tubes or conduits, as well as the chambers of the exhaust pathogen degradation unit.

It is also noted that the inhalation treatment unit and the exhalation treatment unit can each define a fluid passage that enables the flow of air from the atmosphere, to the mask for inhalation, and then back to the atmosphere; and the fluid passage can be equipped with anti-return valves to prevent backflow of the air. The anti-return valves can be positioned at certain locations along the fluid passage to ensure functioning of the treatment units and to enhance safety of the user. The inhalation treatment unit can also include a pressurization system for pressurizing the air to force it to flow through the unit to the mask.

Either one of the inhalation treatment unit and the exhalation treatment unit, or both, can include one or more filtration units. The filtration unit includes a filter that is made of a porous material configured to retain, or trap, various contaminants therein such as pathogens and particles, or any other types of contaminants that can be retained via filtration mechanisms. In some implementations, the filter can be removed from its operational location, i.e., the location where the filtration unit is installed to fulfill its function of filtration, to be cleaned and decontaminated. In other words, the filter can be removed from the inhalation treatment unit or the exhalation treatment unit by the user to be cleaned and decontaminated. Once decontaminated, the filtration unit can then be repositioned to its operational location by the user and re-used for filtration. Alternatively, the filter can remain in its operational location to be cleaned and decontaminated, either when the mask is in use or when the mask is not in use. The filter can thus be subjected to a filtration stage and a cleaning stage, either sequentially or simultaneously, for repetitive usage. Accordingly, the filtration unit can be used to perform filtration for a certain period of time, be removed from the inhalation treatment unit or the exhalation treatment unit to be cleaned, and then be placed back into the corresponding inhalation treatment unit or exhalation treatment unit to perform filtration. The filtration unit can also be used to perform filtration and be cleaned at the same time such that contaminants retained in the filter are being deactivated. The filtration unit can also remain at its operational location although not in use by the user, and be subjected to cleaning and decontamination prior to being ready for re-use by the user. The choice of scenario can depend on the type of decontamination being performed during the cleaning stage.

The filtration unit can also be integrated into a mask wearable on the user's face, in contrast to when the filtration unit is provided separately while being in fluid communication therewith, such as when the filtration unit is included in the inhalation treatment unit or the exhalation treatment unit as mentioned above. When the filtration unit is integrated into the mask, ambient air to be inhaled by a user as well as subsequently exhaled air from the user can both be treated by the filtration unit to retain contaminants therein.

Various implementations of the multistage treatment system will now be described in greater detail.

### Inhalation Treatment Unit

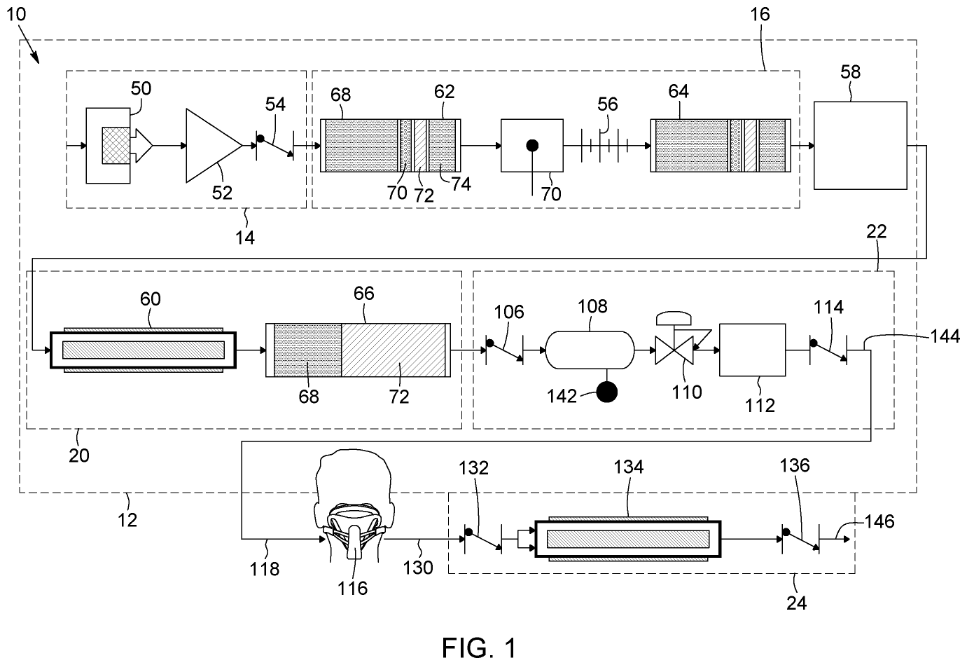

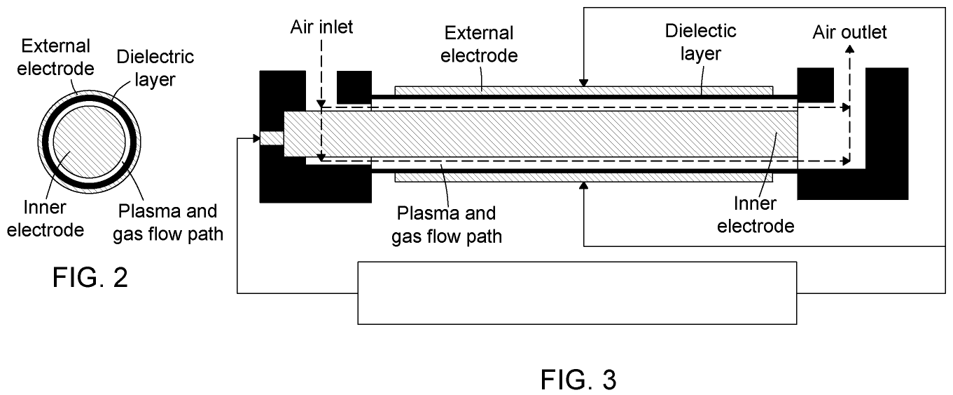

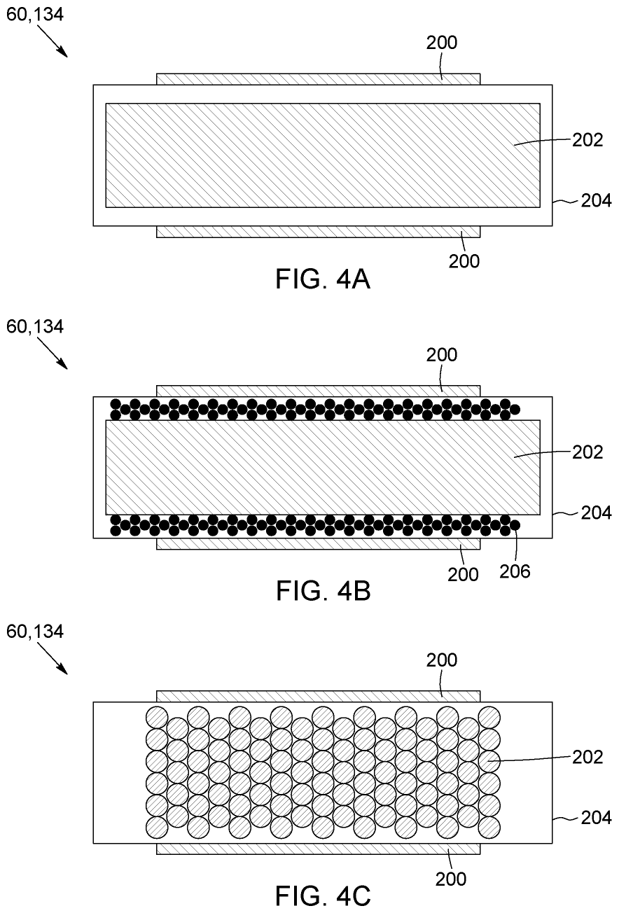

With reference to FIGS. 1, 7, 17A and 17B, examples of a multistage treatment system 10 that includes an inhalation treatment unit 12 are shown. In some implementations, the inhalation treatment unit 12 includes an air intake section 14, a thermal treatment section 16, a UV treatment section 18, a plasma reactor section 20, and a pre inhalation treatment section 22. Each of these components of the inhalation treatment unit will now be described in further detail.

### Air Intake Section

Referring to FIG. 1, the air intake section 14 is configured to receive a flow of ambient air, which may be contaminated by various contaminants (e.g., particles, gases, microbial pathogens). The air intake section includes an inlet, a particle filter 50, an air pump 52 (also referred to as an air compressor) and a check valve 54 (also referred to as an anti-return valve). The particle filter 50 is configured to separate particles from the air and produce a particle-reduced stream. The air pump 52 drives the flow of air through the air intake section 14 via the inlet and then through the particle filter 50 and also through the inhalation treatment unit 12 in general. In some implementations, the pump 52 can be a diaphragm pump. In some implementations, the pressure can be raised up to between about 100 kPag and about 150 kPag. In some implementations, the pressure can be raised for instance to about 600 kPag. More details regarding the pressurization of the multistage treatment system upstream of the pressure regulator are provided below. The check valve 54 ensures a unidirectional flow of the particle-reduced stream. In other words, the check valve 54 allows a forward flow of the pressurized particle-reduced stream while preventing its backflow into an upstream portion of the air intake section 14. Various types of check valve can be used in the air intake section 14. In some implementations, given the pressurization of the system, check valves made of metals can be chosen to take advantage of their robustness. The check valve 54 can also be made of other materials suitable for a pressurized application. In some implementations, the check valve 54 can be for instance a ball check valve. The particle filter 50 can be any filter suitable to remove particles or particulate matter having a diameter. In some implementations, the particle filter is configured to remove particles having a diameter of up to about 100 microns.

In addition, while the illustrated embodiment shows the particle filter 50 followed by the air pump 52 and then the anti-return valve 54, it should be noted that various other arrangements are possible. It is also noted that other units could also be added to the air intake section 14, such as other removal units to remove contaminants prior to the subsequent stages of the inhalation treatment unit. It is also noted that the air intake section 14 may not have a filter or other removal unit, but may simply include an air pump and optionally an anti-return valve depending on the subsequent unit and whether flow return should be avoided.

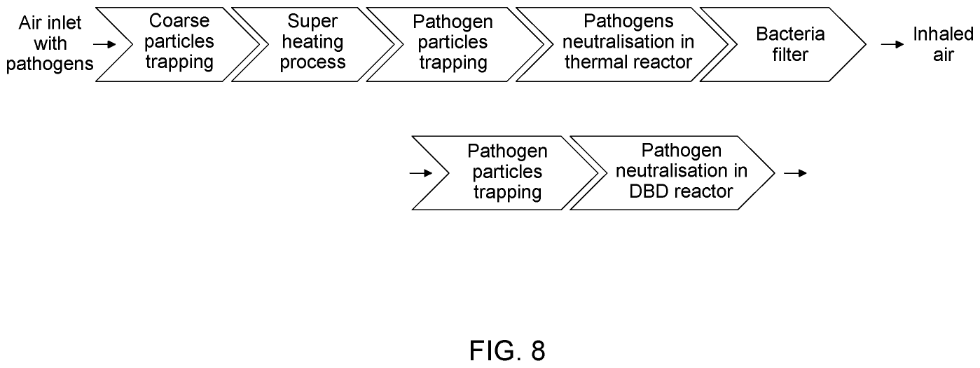

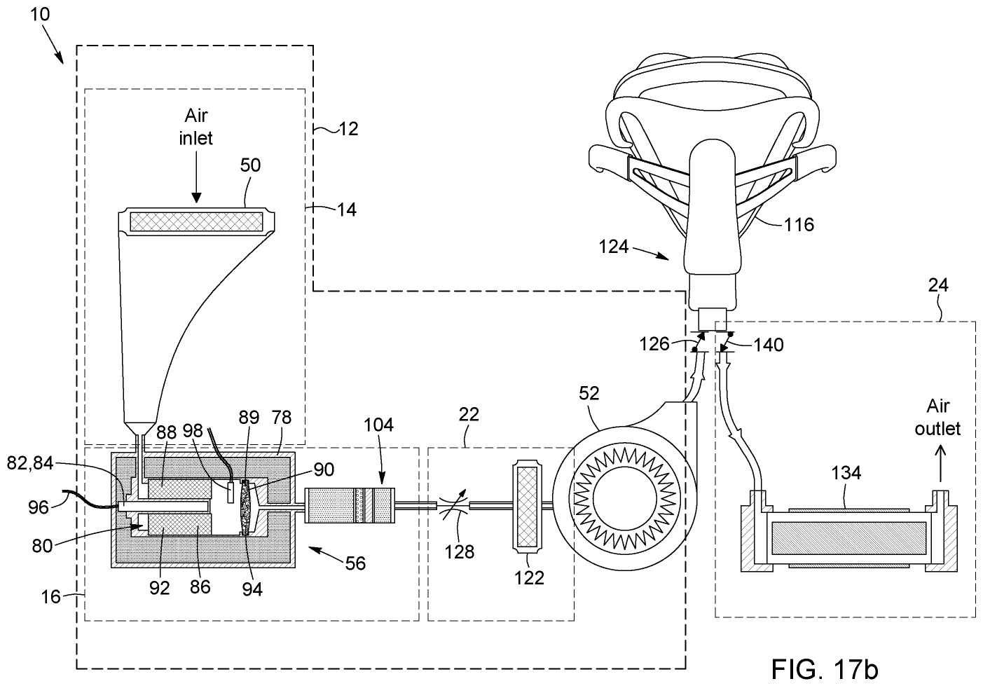

In some implementations and with reference to FIGS. 7, 17A and 17B, the air intake section 14 can include an inlet, a particle filter 50, and an air pump 52, which can also be referred to as a “blower”. As mentioned above, the particle filter 50 can be configured to separate particles from the air and produce a particle-reduced stream. The air pump 52, or blower, is configured to drive the flow of air to be treated through the air intake section 14 via the inlet and then through the particle filter, and to supply the air to the thermal treatment section. In some implementations and as shown in FIG. 17A, the air pump 52 can be provided as part of the air intake section 14 to drive the flow of air into downstream sub-units of the inhalation treatment unit 12. In other implementations and as shown in FIG. 17B, the air pump 52 can be provided as part of the pre-inhalation treatment section 22, and be configured to suction in the flow of air into upstream sub-units of the inhalation treatment unit 12. In other words, the air pump 52 can be configured to suction in the contaminated air into the air intake section 14 and then into the sub-units provided downstream of the air intake section 14. In some implementations and for instance in the implementations shown in FIGS. 7 and 17A, the air pump 52, or blower, can be configured to pressurize the air to be treated above atmospheric pressure.

The air intake section 14 is in fluid communication with one or more pathogen degradation units which receives the pressurized and optionally particle-reduced stream.

### Pathogen Degradation Unit(s)

The inhalation treatment unit 12 further includes one or more pathogen degradation units located downstream of the air intake section 14. The pathogen degradation unit, or a first one of the pathogen degradation units, is configured to receive the pressurized air or suctioned air, and reduce its pathogen content.

FIG. 1 illustrates an implementation of the inhalation treatment unit 12 that includes three pathogen degradation units: a heating unit 56, a UV treatment unit 58, and a plasma reactor unit 60. Each of these units is integrated in a corresponding section of the inhalation treatment unit 12, which may or may not include additional features and structures.

In FIG. 1, the heating unit 56 is included in a thermal treatment section 16, which further includes a first contaminant removal unit 62 provided upstream of the heating unit 56, and a second contaminant removal unit 64 provided downstream of the heating unit 56. The UV treatment unit 58 is provided in a UV section 18. The plasma reactor unit 60 is included in a plasma reactor section 20, which further includes a third contaminant removal unit 66 downstream of the plasma reactor unit. Details regarding each of these sections are provided below.

In the implementations illustrated in FIGS. 7 and 17, the inhalation treatment unit 12 includes is a heating unit 56 as a pathogen degradation unit. The heating unit 56 is included in a thermal treatment section 16, which may or may not include additional features and structures.

Thermal Treatment Section

The thermal treatment section 16 is in fluid communication with the pressurized air intake section 14 described above. The pressurized particle-reduced stream is directed to the first contaminant removal unit 62 (which can also be referred to as a “trap” or filter) to remove contaminants such as water vapour, CO₂, hydrocarbons (e.g., non-methane hydrocarbons (NMHC)), VOCs and halogen compounds, if any are present. The first contaminant removal unit 62 can include different layers each having a corresponding purpose to increase the overall efficiency of the contaminant removal process. For instance, the first contaminant removal unit 62 can include a molecular sieve, a desiccant, activated charcoal, or any other suitable adsorbent. In the implementation shown in FIG. 1, the first contaminant removal unit 62 includes four layers: a first layer of a molecular sieve 68, for instance a molecular sieve that can be configured to adsorb molecules with effective diameters smaller than 1 nm, a second layer of desiccant 70, a third layer of activated charcoal 72, and a fourth layer of a molecular sieve 74 that can be similar or different from the molecular sieve used in the first layer.

In some implementations, the first contaminant removal unit 62 is configured to reduce the CO₂ content of the air below a given threshold. Reducing the CO₂ content can be beneficial in one or more later stages of the inhalation treatment unit 12, e.g., to avoid the undesirable conversion of CO₂ to CO for instance during treatment in the plasma reactor unit 60. It may be desirable to avoid CO production because this byproduct should not reach the user for inhalation and yet is relatively difficult to remove if formed. Thus, by removing its precursor, CO formation and challenges related to it can be mitigated.

In some implementations, the first contaminant removal unit 62 can contribute to reducing the velocity of the air stream, which can facilitate increasing the residence time of the air in the thermal treatment section.

The air stream exits the first contaminant removal unit 62 and flows into a heating unit 56 to be thermally treated at a temperature sufficient to reduce a pathogen content of the air and produce a thermally treated stream. In some implementations, the thermal treatment can be operated at temperatures between about 250° C. to about 350° C. In some implementations, the temperature at which the thermal treatment is operated as well as the residence time of the air stream in the heating unit is sufficient to kill or deactivate a majority of or substantially all germs, such as viruses and bacteria, that may be contained in the air stream.

The temperature of the thermal treatment can be monitored using a temperature sensor 76, and a controller operatively connected to the heating unit 56. When the temperature sensor 76 detects a temperature below a given lower threshold, the controller can turn on or heat up the heating unit 56, and when the temperature sensor 76 detects a temperature above a given higher threshold, the controller can turn the heating unit 56 off or down. In some implementations, the lower threshold can be between about 225° C. to about 275° C., while the higher threshold can be between about 325° C. and about 350° C.

The heating unit 56 can include a heating vessel that can be made of various materials. In some implementations, a heating element is inserted into the heating vessel. The heating element can be a cartridge heater. A filler such as a metal mesh or metal wool can also be inserted in the heating vessel to improve thermal destitution and increase the surface area. Examples of suitable fillers can include a stainless steel wool or stainless steel mesh, or a copper wool or copper mesh.

The heating vessel can be insulated with an insulation material to reduce heat losses. The insulation material can be for instance a mineral-based insulation material, ceramic fiber insulation material, and perlite insulation material. In some implementations, the heating vessel can be heated by an ethanol gel, butane, or an electrical heating system.

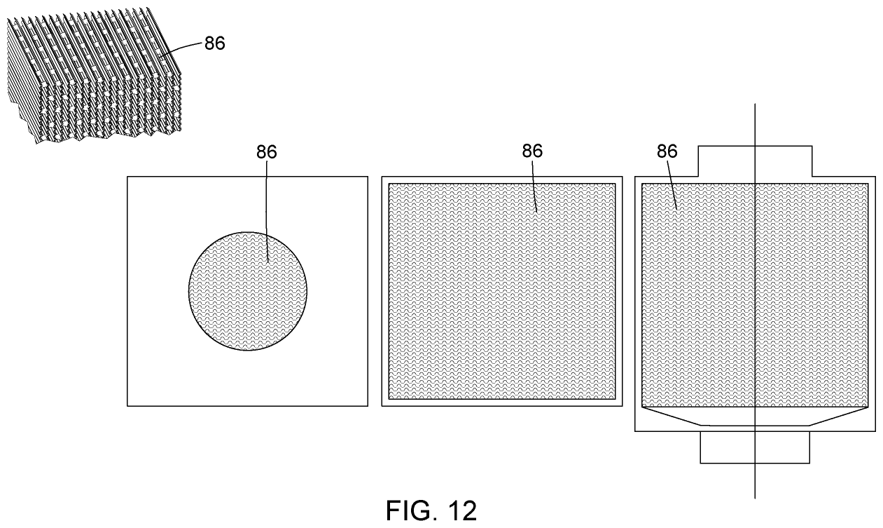

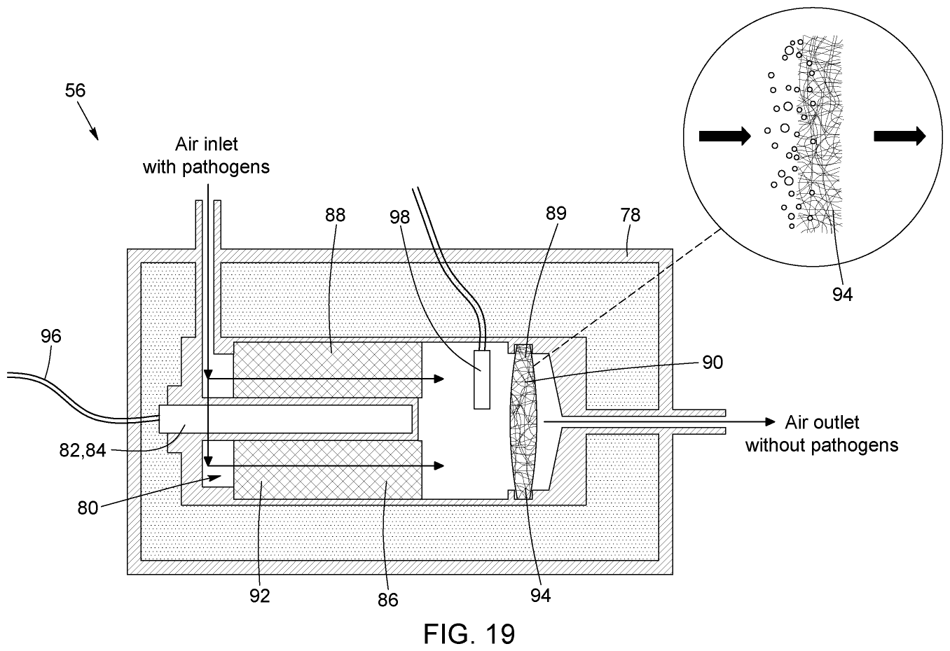

With reference to FIGS. 7, 11 and 12, when the heating unit 56 includes a heating vessel 78, for instance a heating vessel made of metal, the heating vessel 78 can include a heating chamber 80 configured to receive one or more pathogen removal units 88 therein. In the implementation shown in FIGS. 7 and 11, the heating element 82 is provided outside the heating chamber 80 in close proximity thereof and within the heating vessel 78, and is operatively connected to a battery (not shown) to provide power to the heating element 82. In some implementations and as mentioned above, the heating element 82 can be provided within the heating vessel 78, i.e., in the heating chamber 80. For instance, the heating element 82 can be a cartridge heater located in the heating chamber 80. FIGS. 17A, 17B and 19 illustrate an implementation where the heating element 82 includes a cartridge heater 84 provided within the heater chamber 80 of the heating vessel 78. In the implementation shown, the cartridge heater 84 is surrounded by a heat dissipator 86, or heat exchanger, which will be discussed in more detail below. Any other suitable heating means can also be used.

In some implementations and as shown in FIGS. 7, 17A and 17B, the first contaminant removal unit can be omitted.

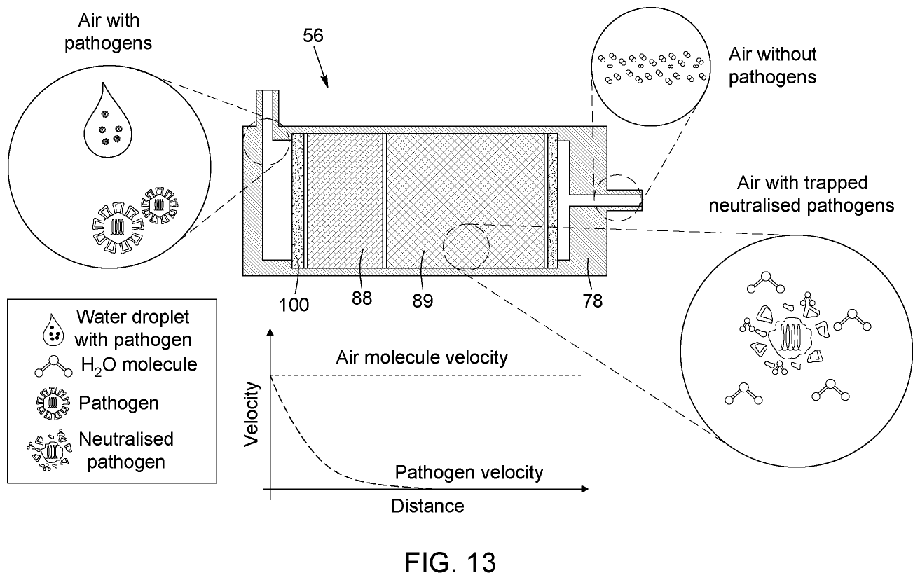

The pathogen removal unit 88 can be made of a porous material that increases the surface area through which the air containing pathogens flows. In some implementations, the porous material can have large pores and be configured as a heat dissipator, or heat exchanger. The heat dissipator can be made of metal, or another thermal conductor material. For instance, FIG. 12 illustrates a heat dissipator 86 received within a heating chamber 80 of a heating unit 78. In some implementations, the heat exchanger can contribute to reducing pressure loss as the flow of air travels therethrough, in comparison with a porous material having smaller pores. The heat exchanger is configured to enhance heat transfer between the air to be treated and the heat provided by the heating element 82, so as to rapidly and efficiently increase the temperature of the air to be treated. In such implementations, the rapid heat transfer can contribute to exposing the pathogens contained in the air to be treated to high temperatures rapidly, which in turn can provide an efficient way to deactivate or destroy the pathogens within the heating chamber 80.

In some implementations, the porous material of the pathogen removal unit 88 can have a pore size that is configured to trap, or retain, pathogens therein. For instance, the pore size for such porous material can range for instance from about 1 nm to 400 nm. In some implementations, the pore size can range from 10 nm to 300 nm. Examples of materials having a pore size in that range can include a molecular sieve or porous glass, such as Controlled Pore Glass (CPG).

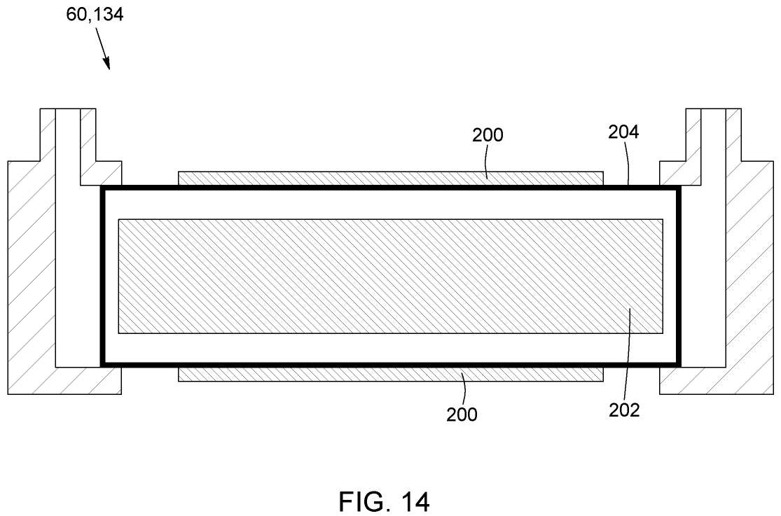

In implementations where the porous material of the pathogen removal unit 88 has a pore size that is configured to trap pathogens therein, the air supplied to the heating unit 56 follows a tortuous path while travelling via the pores through the pathogen removal unit 88, while pathogens having a size larger than the size of the pores become trapped in the pathogen removal unit 88. Pathogens being trapped in the pathogen removal unit 88 thus remain in the heating unit 56 for an increased residence time, which in turn, can contribute to deactivating or destroy pathogens initially contained in the air as pathogens are exposed to heat for a longer period of time. This concept is illustrated schematically in FIG. 13, which shows that the velocity of the air flow through a first and a second successive pathogen removal units 88 remains substantially similar while travelling therethrough. In contrast, the velocity of the pathogens is significantly reduced as they are introduced in the pathogen removal units 88, as the pathogens are being trapped in the porous material of the pathogen removal units 88. Of course, FIG. 13 is for illustrative purposes only, and it should be understood that the velocity of the air flow travelling through the heating unit 56 can also be reduced depending on the pore size of the porous material.

In some implementations, pathogens can be associated with water droplets contained in the air to be treated, and the tortuous path provided by the pathogen removal unit 88 can facilitate water droplets to vaporize, thereby releasing pathogens therefrom. The pathogen removal unit 88 can then trap pathogens that were once associated with the vaporized water and that are now dissociated therefrom. Concurrently, as heat is provided to the heating unit 56, the pathogens trapped in the pathogen removal unit 88 are exposed to a temperature that is sufficient to deactivate or destroy pathogens.

In some implementations, the temperature at which the thermal treatment is conducted can range for instance from about 100° C. to about 450° C. In some implementations, the temperature at which is conducted the thermal treatment is sufficient to superheat water contained in the air to be treated. In addition to deactivating or destroying pathogens by the application of heat, superheating the water droplets contained in the air to be treatment can lead to the formation of byproducts than can in turn have an impact on the viability of pathogens. For instance, the increased concentration of hydroxide ions (OH⁻) and hydronium ions (H₃O⁺) in superheated steam can lead to oxidative reactions taking place within the pathogen removal unit 88 to further contribute to deactivating or destroy pathogens. Furthermore, ions present on the surface of the porous material, such as Ca²⁺ and Na⁺, can act as catalysts to break down organic molecules that may be present in the air to be treated. The combination of the heat provided to the heating unit 56 and the use of a pathogen removal unit 88 thus provides a means to dissociate pathogens from water droplets, to trap the pathogens within the pathogen removal unit 88, and to deactivate or destroy the trapped pathogens by the application of heat, and in some implementations, via various chemical reactions occurring within the pathogen removal unit(s) 88.

In some implementations, when more than one pathogen removal unit 88 is received within the heating chamber such as shown in FIG. 7, a first pathogen removal unit 88 can be configured to receive the particle-reduced stream and provide a first porous region having an increased surface area for vaporizing water present in the contaminated air and removing vapour contaminants therefrom, and a second pathogen removal unit 88 can also be received within the heating chamber and be configured to provide a second porous region having also an increased surface area and vaporize water present in the contaminated air to further remove vapour contaminants therefrom. In the context of the present description, the expression “vapour contaminant” can include pathogens. The second pathogen removal unit 88 can contribute to further increase the residence time of pathogens contained in the air in the heating unit. In some implementations, the thermal treatment can be conducted at a temperature sufficient to produce superheated steam which, as mentioned above, can be beneficial for deactivating or destroying pathogens. For instance, the first pathogen removal unit 88 can be made of a material having a first pore size, and the second pathogen removal unit 88 can be made of a material also having a second pore size, different than the first pore size. In some implementations, the first pore size can be smaller than the second pore size, or vice versa. The choice of material and of the pore size can depend on the pathogens that are desired to be removed from the air, and the resulting residence time of the pathogens within the pathogen removal unit 88(s) once in use.

In some implementations, a pathogen removal unit 88 configured as a heat dissipator 86 can be provided in combination with a pathogen removal unit 88 having pores that are small enough to trap pathogens therein, either inside the heating chamber 80 or outside the heating chamber 80. For instance, a heat dissipator 86 can be provided upstream of a pathogen removal unit 88 having pore small enough to trap pathogens therein. Such a configuration can contribute to raising the temperature of the air to be treated rapidly and deactivate or destroy at least some of the pathogens contained in the air to be treated. The subsequent pathogen removal unit(s) 88 having pores that are small enough to trap pathogens can then provide an additional opportunity to further deactivate or destroy remaining pathogens by trapping and exposing them to heat.

FIGS. 17A, 17B, and 19 illustrate an example of a heating unit 56 that includes a heating vessel 78 defining a heating chamber 80. A cartridge heater 84 is received into the heating chamber 80 to supply heat thereto. The cartridge heater 84 is operatively connected to a power source, such as a battery. The cartridge heater 84 is surrounded by a heat dissipator 86, or heat exchanger. Further received into the heating chamber 80 is a porous material 90, which is shown as being located downstream of the heat dissipator 86. In the implementation shown, two pathogen removal units 88 are thus received within the heating chamber, i.e., the heat exchanger 86 as a first porous region 92, and the downstream porous material 90 as a second porous region 94 (the usage of the terms “first” and “second” is to facilitate the reference to the porous regions when more than one porous region is present, and can be used interchangeably).

A first temperature sensor 96 can be provided to monitor the temperature in the vicinity of the heating element, which in the implementation shown in FIGS. 17A, 17B and 19 is represented as a cartridge heater 84. A second temperature sensor 98 can be provided in the vicinity of the second porous material 94 to monitor the temperature in the area surrounding the second porous material 94. As will be explained in further detail below, monitoring the temperature within the heating chamber 80 in the vicinity of the second porous material 94 can contribute to ensure that a temperature sufficiently high is reached for a given period of time to deactivate and destroy pathogens that may accumulate within the porous material, and in particular in the second porous material 94. The monitoring of temperatures can also be beneficial to implement a given heating cycle, or pathogen removal cycle, as will also be discussed in further detail below.

The heating vessel 78 is in fluid communication with the air intake section 14 and is configured to receive the contaminated air, which may or may not have been subjected to filtering to produce a particle-reduced stream. The air to be treated then travels through the heat dissipator 86, or heat exchanger. As mentioned above, the heat dissipator 86 is configured to enhance heat transfer between the air to be treated and the heat provided by the heating element to rapidly and efficiently increase the temperature of the air to be treated within the heating chamber 80, which can contribute to expose the pathogens contained in the air to be treated to high temperatures rapidly. In some implementations, the heat exchanger can include a metal mesh or metal wool, such as a stainless steel wool or stainless steel mesh, or a copper wool or copper mesh. The porous material illustrated in FIG. 12 can be an example of metallic mesh that can be suitable for use as for the heat dissipator 86 of the implementation shown in FIGS. 17A, 17B and 19. In some implementations, the porous material of the heat dissipator 86 can have a pore size a pore size that is configured to trap, or retain, therein pathogens having a larger size than the pore size. In some implementations, the heat dissipator 86 can be considered a pathogen removal unit 88. Trapping pathogens within the heat dissipator 86, which can be a first porous material, can contribute to heat pathogens at a temperature sufficiently high and/or for a sufficiently long period of time to deactivate or destroy the pathogens. In other implementations, the heat dissipator 86 can also have large pores and be configured to heat the air to be treated rapidly and efficiently.

Still referring to FIGS. 17A, 17B and 19, the second porous region can include a metal network, or a series of metal network. The metal network can also be referred to as a metal mesh or a porous metal fibre particulate filter. The metal mesh can be made for instance of a stainless steel mesh. The metal mesh can be configured to as to filter pathogens by both surface and depth filtering. In some implementations, the metal mesh can have pores of less than about 10 nm. In some implementations, the metal mesh can have pores of between about 1 nm and about 10 nm. In some implementations, the metal mesh can have pores of less than about 1 nm. In some implementations, the pore size of the metal mesh can be determined such that substantially no pathogens can travel therethrough, the pore size of the metal mesh being smaller than the size of the pathogens to be removed from the air to be treated. In such implementations and as mentioned above, the pathogens having a larger size than the pore size of the porous material can accumulate within the porous material for a given period of time. As the pathogens remain trapped within the second porous region, the pathogens can be exposed to heating at temperatures sufficiently high to be deactivated or destroyed.

In some implementations, the metal mesh, or porous metal fibre particulate filter, can be made of sintered metal fibers. In some implementations, one or more layers of sintered metal fibers can be present to form the metal mesh, or porous metal fibre particulate filter. The metal fibers can be configured to lay substantially uniformly to form a three-dimensional non-woven structure, that can optionally be sintered at contact points. In implementations where multiple layers are present, the metal mesh, or porous metal fibre particulate filter, can use different metal fibre sizes for the different layers, which can contribute to enhance filtering performance. The presence of multiple layers of metal fibers can contribute to improve filtering efficiency and can allow to trap small pathogens, for instance those having a diameter of less than about 1.5 nm, while maintaining a small, or very small, pressure drop therethrough. In turn, reducing the pressure drop across the metal mesh can reduce the power consumption of the portable device, for instance by reducing the power consumption of the pump, or blower. In addition, the thermal conductivity of the metal mesh can be beneficial to enhance heat transfer by providing substantially uniformly distributed heat, and in turn the heat can contribute to neutralise or deactivate pathogens.

It is to be noted that in some implementations, a pathogen removal unit 88 can include a plurality of metal networks (metal mesh or a porous metal fibre particulate filter) provided in series. The plurality of metal networks can be provided for example in an adjacent relationship or in a spaced-apart relationship. For instance, more than one metal network as shown in FIGS. 17A and 17B can be provided in series. In some implementations, providing a series of metal networks can contribute to increase the volume of the pathogen removal unit 88.

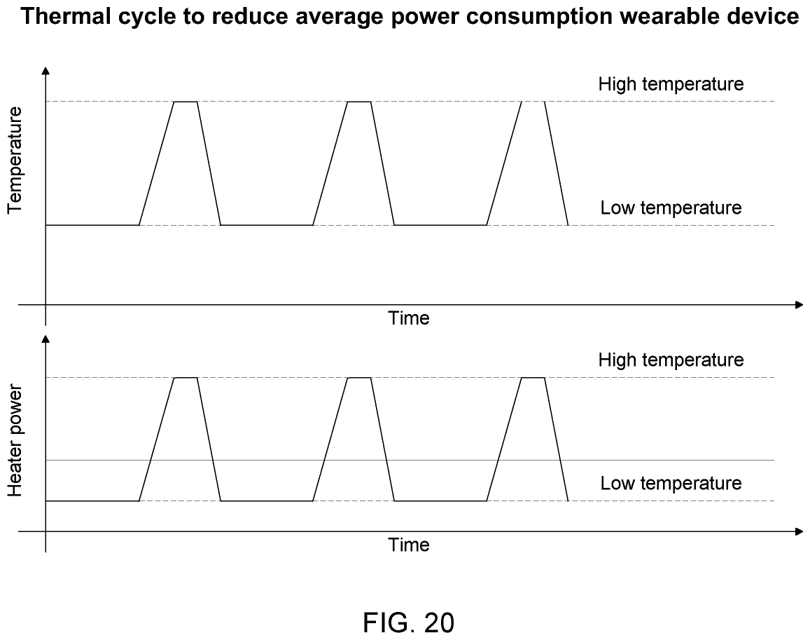

With reference to FIG. 20, in some implementations, the temperature within the heating chamber 80 can be controlled according to a given heating cycle. The heating cycle can be beneficial for instance to reduce power consumption and can contribute to prolonging battery life. The heating cycle can result from the alternance between the temperature monitored within the heating chamber 80 and obtained according to a low temperature setpoint, or low temperature threshold, and according to a high temperature setpoint, or high temperature threshold. In some implementations, the low temperature setpoint can be set at a temperature, or within a range of temperatures, that is still sufficiently high to deactivate or destroy pathogens. For instance, the low temperature setpoint can be set between about 150° C. to about 200° C., between about 200° C. and about 300° C., or between about 250° C. and 350° C. Alternatively, in some implementations, the low temperature setpoint can be set at a temperature that is lower than about 150° C. The high temperature setpoint is set at a higher temperature than the low temperature setpoint, and can corresponds to a temperature that is sufficiently high to deactivate or destroy pathogens. In some implementations, the high temperature setpoint can be set between about 225° C. to about 275° C., between about 275° C. and about 350° C., or between about 350° C. and about 400° C. It is to be noted that the ranges given for the low temperature setpoint and the high temperature setpoint are examples to illustrate the general principle of the heating cycle, but that other temperature setpoints can also be suitable. In some implementations, the temperature setpoints can be set according to the type(s) of pathogens to be destroyed.

The power supplied to the heating element can alternate between a low power setpoint, or low power threshold, and a high power setpoint, or high power threshold, resulting in an alternance of periods of low power and high power according to a given sequence, which can correspond to the alternating sequence of the low temperature setpoint and the high temperature setpoint.

Still referring to FIGS. 17A, 17B and 19, a controller can be operatively connected to the heating element 82, which can be a heater cartridge 84, and to the temperature sensor 96. The controller can be configured to provide the necessary power to the heating element such that in turn the heating element 82 provides the heat required to reach the temperature setpoint according to the heating cycle, which can be either the low temperature setpoint or the high temperature setpoint. The given temperature setpoint will then be maintained for a given period of time. Then, once the given period of time has ended, the low temperature setpoint can be switched to a high temperature setpoint (or the high temperature setpoint is switched to a low temperature setpoint) for another given period of time, and so on.

As mentioned above, implementing such a heating cycle can be beneficial to reduce overall power consumption, which can advantageously prolong battery life. In addition, in some implementations, operating the heating element 82 between a low temperature setpoint and a high temperature setpoint can enable to perform a pathogen removal cycle that can include at least two phases. In a first phase of the pathogen removal cycle, the pathogens contained in the air to be treated can accumulate within the second porous material 94 while the temperature is set at the low temperature setpoint. As pathogens are trapped within the pores of the second porous material 94 and accumulate therein, the pathogens are subjected to temperatures according to the low temperature setpoint, which may be sufficient to deactivate or destroy at least a portion of the pathogens. After the certain period of time at this low temperature setpoint, which can be said to correspond to a certain residence time of the pathogen within the second porous material, the temperature can be increased to the high temperature setpoint in a second phase of the pathogen removal cycle. In this second phase of the pathogen removal cycle, temperatures can be sufficiently high to ensure that substantially all of pathogens trapped in the second porous material 94 is destroyed.

In some implementations, the repetition of the first and second phase of the pathogen removal cycle over time can ensure that as a continuous flow of air to be treated enters the heating unit 56, pathogens trapped in the second porous material 94 can be destroyed. In some implementations, the duration of the first phase of the pathogen removal cycle can be similar to the duration of the second phase of the pathogen removal cycle. In other implementations, the duration of the first phase of the pathogen removal cycle can be longer compared to the duration of the second phase of the pathogen removal cycle. A longer first phase of the pathogen removal cycle can be advantageous to reduce the power supplied to the heating element 84 for a longer period of time, while the duration of the second phase of the pathogen removal cycle is sufficient to destroy substantially all pathogens trapped in the second porous material 94, concomitantly with the high temperature setpoint. In some implementations, the duration of the first phase of the pathogen removal cycle can be for instance one or two hours, and the duration of the second phase of the pathogen removal cycle can be for instance fifteen or thirty minutes. Again, these durations are examples to illustrate the general principle of the pathogen removal cycle, and it is to be understood that other durations can also be applicable.

The thermal treatment section can also include a cooling unit to reduce the temperature of the thermally treated stream. In some implementations, the cooling unit can be, for instance, an atmospheric heat sink. In some implementations and with reference to FIG. 7, the cooling unit can include a heat exchanger comprising tubing 102 to cool down the treated air. The tubing 102 can be made for instance of metal or plastic, and can have a diameter ranging from 1 cm a 5 cm. Reducing the temperature of the thermally treated stream can be beneficial for the operation of one or more downstream removal or treatment steps. In some implementations, the temperature of the thermally treated stream can be reduced close to ambient temperature (e.g., about 20° C.).

The thermally treated air is directed to a second contaminant removal unit 64 configured to further remove vapour contaminants therefrom if present. These contaminants can include water vapour, CO₂, VOC, hydrocarbons and/or halogen compounds. In FIG. 1, the configuration of the second contaminant removal unit 64 is similar to the configuration of the first contaminant removal unit described above. The illustrated example of the second contaminant removal unit 64 thus includes a first layer of a molecular sieve 68, a second layer of desiccant 70, a third layer of activated charcoal 72, and a fourth layer of a molecular sieve 74 that can be similar or different from the molecular sieve 68 used in the first layer. It is noted that this second contaminant removal unit 64 can be different from the first contaminant removal unit 62, including different materials, layers, and arrangements for contaminant removal.

In some implementations, providing a first contaminant removal unit 62 upstream of the heating unit 65 and a second contaminant removal unit 64 downstream of the heating unit 65 can provide certain advantages. For instance, the first contaminant removal unit 62 can facilitate the removal of selected contaminants such as hydrocarbons, CO and CO₂ upstream of the heating unit 56 to avoid formation of undesirable byproducts during the thermal treatment. The second contaminant removal unit 64 can then be provided to remove byproducts such as CO₂, N₂O or NOx, if generated during the thermal treatment.

In some implementations and as illustrated in FIGS. 7, 17A and 17B, the thermally treated air can be directed to a contaminant removal unit 104 configured to remove byproducts that may have been produced during the thermal treatment or other contaminants. For instance, in implementations where superheated steam was produced during the thermal treatment, it may be desirable to remove contaminants associated with the superheated steam. The contaminants can also depend on the composition of the air to be treated. Examples of contaminants can include for instance water vapour, VOCs, hydrocarbons, OH⁻, O₃, N₂O, CO, NOx and CO₂. In FIGS. 7, 17A and 17B, the configuration of the contaminant removal unit 104 is similar to the configuration of the first or second contaminant removal unit 62, 64 described above, and includes a first layer of a molecular sieve 68, a second layer of desiccant 70, a third layer of activated charcoal 72, and a fourth layer of a molecular sieve 74 that can be similar or different from the molecular sieve used in the first layer. Other configurations of the contaminant removal unit 104 than the one illustrated in FIGS. 7, 17A and 17B are possible, and can include for instance different materials and additional layers or fewer layers.

UV Treatment Section

The thermal treatment section 16 is in fluid communication with the UV treatment section 18. The UV treatment section 18 includes a UV treatment unit 58 having a UV chamber configured to receive and house the thermally treated air, and a UV light source. The UV chamber can include a tube made of a UV transmitting material, such as quartz or sapphire. The light source is configured to emit UV radiation to contact the air within the UV chamber and produce a UV treated stream. The UV light source can be for instance a UV LED or a 254 nm UV lamp. Contacting the thermally treated stream with UV radiation notably kills or deactivate pathogens that may be present.

In some implementations, the UV treatment section 18, e.g., the UV treatment unit 58, can be pressurized to increase the residence time of the air in the UV chamber. The pressure in the UV treatment section can be set according to the buffer tank pressure set point, which determines the pressure in the multistage treatment system upstream of the pressure regulator.

Plasma Reactor Section

The UV treatment section 18 is in fluid communication with the plasma reactor section 20. In the implementation illustrated in FIG. 1, the plasma reactor section 20 includes a coaxial plasma reactor 60 that includes an inlet, a plasma chamber, a dielectric layer, plasma-generating mechanism, and an outlet. The plasma chamber of the plasma reactor 60 includes a gas flow path allowing a flow of the air (e.g., the UV treated stream) to flow through the plasma chamber from the inlet to the outlet. The plasma-generating mechanism is configured to apply a plasma-generating field across the plasma chamber intersecting the flow of the air stream.

The plasma-generating mechanism can include an internal electrode and an external electrode positioned in a concentric configuration relative to each other. In some implementations, the external electrode and the internal electrode can be operatively connected to a power supply providing an AC current. In some implementations, the plasma-generating mechanism relies on a dielectric-barrier discharge (DBD). In other implementations, other type of discharges could be used such as a direct-current plasma (DCP) or corona discharge. In some implementations, the coaxial section of the coaxial plasma reactor where the gas flows via the gas flow path can contain spheres or other material to increase surface area and reduce flow velocity. The spheres can be for example glass spheres. In some implementations, the inner electrode can be made of electrically conductive spheres of material. As such, the quartz tube of the plasma reactor can be filled with those electrically conductive spheres to increase reactive surface and plasma while reducing velocity of the flow of gas.

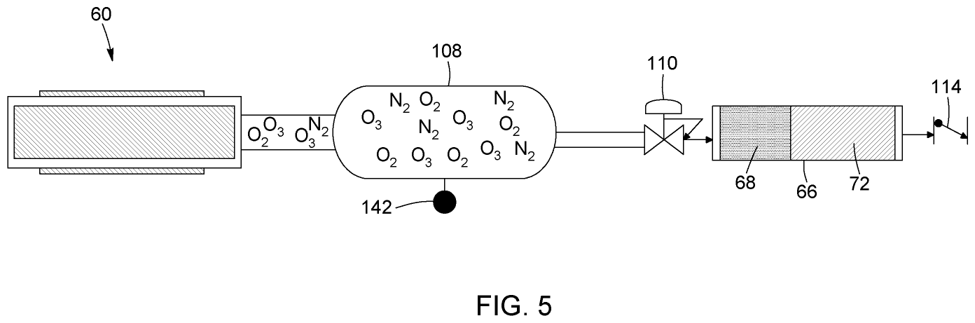

Examples of various configurations of the plasma reactor are shown in FIGS. 4A-4C. FIG. 4A illustrates an implementation of a plasma reactor 134 having a pair of external electrodes 200, a conductive inner electrode 202, and a dielectric shell 204. FIG. 4B illustrates an implementation of a plasma reactor 134 that includes a pair of external electrodes 200, a conductive inner electrode 202, a dielectric shell 204, and dielectric beads 206. FIG. 4C illustrates an implementation of a plasma reactor 134 that includes a pair of external electrodes 200, a conductive inner electrode 208 that is made of beads, and a dielectric shell 204.

Flowing the air stream through the plasma chamber and subjecting the air to the plasma-generating field generates a plasma, thereby producing a plasma treated stream that includes plasma-generated compounds. Plasma-generated compounds can include CO, N₂O, NOₓ and ozone, which may be present in trace amounts. In some implementations, it may be desirable to remove such plasma-generated compounds from the plasma treated stream to avoid the used to inhale such compounds. As noted above, it can also be of interest to pre-treat the air to remove precursors that would be transformed into plasma-generated compounds, e.g., removing CO₂ to reduce or avoid CO formation.

The plasma reactor section may thus include a third contaminant removal unit 66 to remove at least a portion of at least some plasma-generated compounds, as shown in FIG. 1. In such implementations, the plasma treated stream is directed to the third contaminant removal unit 66 to produce a stream of treated air. In FIG. 1, the third contaminant removal unit 66 includes two layers: a first layer that includes a molecular sieve 68, and a second layer that includes activated charcoal 72. Other configurations of the third contaminant removal unit 66 can be implemented, and the choice of the components of the third contaminant removal unit 66 can depend on the specific plasma-generated compounds that are found in the plasma treated stream. The third contaminant removal unit 66 can be provided to substantially remove N₂O, NOₓ and ozone generated by the plasma reactions.

Viruses that can be destroyed by the present multistage treatment unit include a SARS virus such as SARS-CoV-2, or a MERS virus.

### Pre Inhalation Treatment Section

In some implementations, following treatment of the contaminated air via the various sections of the inhalation treatment unit 12, the plasma treated stream can be supplied to a pre inhalation treatment section 22. Whereas one of the main objectives of the preceding sections (i.e., the air intake section 14, the thermal treatment section 16, the UV treatment section 18 and the plasma reactor section 20) is to reduce the content of a number of contaminants (e.g., particles, chemical compounds and pathogens), one of the purposes of the pre inhalation treatment section 22 is to improve the experience of the user, for instance by increasing the humidity of the treated air and regulating its flow for supply to the mask.

In FIG. 1, the pre inhalation treatment section 22 includes a valve 106, a buffer tank 108 configured to receive the treated air, a pressure regulator 110 coupled to the buffer tank 108, a humidifier 112 coupled to the pressure regulator 110 and receiving the treated air to produce a humidified treated air, and a feed inlet 118 comprising a valve 114, such as an anti-return valve, for supplying the humidified treated air to the mask 116. The valve 114 also ensures that subsequently exhaled air from the user does not backflow into the inhalation treatment unit 12. In some implementation, an additional filter, such as a bacterial-viral filter, can be provided upstream of the humidifier 112.

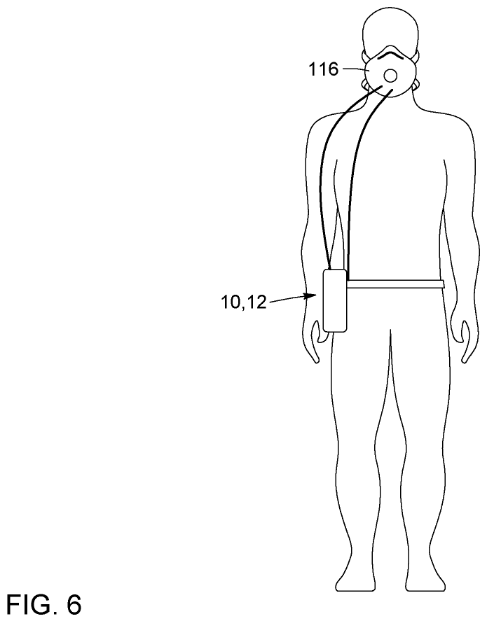

In some implementations, the third contaminant removal unit 66 described above can be positioned downstream of the buffer tank 108 instead of between the plasma reactor unit 60 and the buffer thank 108. This configuration can allow reactive molecules such as ozone to accumulate within the buffer tank 108 and can offer another mean of neutralizing pathogens if present in the treated air, since ozone is a reactive molecule that can destroy pathogens. A representation of this implementation is shown in FIG. 5.

As mentioned above, the buffer tank 108 receives the treated air and also accumulates a certain volume thereof to ensure that the user has access to treated air without interruption. For instance, the buffer tank 108 can house a volume of treated air that may be sufficient to provide the user with treated air for a period of time in the range of seconds or minutes. Housing a volume of treated air in a buffer tank 108, as shown in FIG. 1, can be advantageous for instance if one of the components of the multistage treatment system experiences a malfunction. The volume of air in the buffer tank 108 depends on the pressure of the inhalation treatment unit 12 and thus of the buffer tank pressure set point. A higher pressure set point will allow to store a larger volume of treated air in the buffer tank 108. In some implementations, the volume of treated air in the buffer tank 108 can be for instance 6 to 10 liters. In other implementations, much larger volumes can be stored in the buffer tank 108 if the pressure set point is increased, for instance in industrial applications. A larger volume of the buffer tank in industrial applications also allows to store a larger volume of treated air.

A pressure sensor is provided to monitor the pressure in the buffer tank 108 and in multistage treatment system upstream of the pressure regulator 110. More details regarding this aspect are provided below.

The pressure regulator 110 is configured to have a discharge pressure close or slightly above to atmospheric pression to provide a regulated flow of treated air to the user. The pressure regulator 110 can take the form of a valve that may be adjustable or a fixed flow reducer in the fluid passage. For example, the pressure regulator 110 can be a pressure relief valve such as a one-way respiratory valve. The pressure regulator 110 can be made for instance of plastic, since at this point in the multistage treatment system, the pressure is close to ambient pressure and thus less susceptible to failure. The pressure regulator 110 can also be made of any other suitable material.

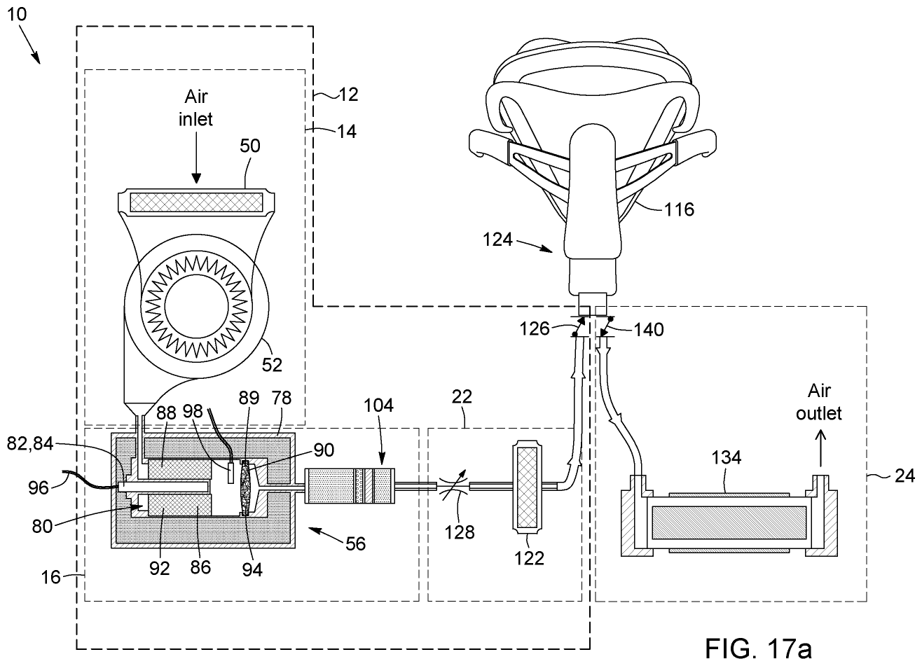

In addition, by providing a pressurized flow above atmospheric pressure to the user, the risk of untreated air infiltration into the mask is reduced. For instance, if the mask does not fit tightly in a sealed manner around the face of the user, the pressurized air mitigates air infiltration through the gaps defined between the mask and the user's face. The positive air pressure also makes it easier for the user to breathe in the treated air, and reduces risks of mask blockage as can be seen in conventional masks such as an N95 mask. FIG. 16 illustrates the effect of providing the flow of treated air above atmospheric pressure when the mask 116 is worn by the user.

As water vapour or a portion thereof may have been removed from the contaminated air during treatment by one or more of the treatment sections of the inhalation treatment unit 12, the humidifier 112 is configured to increase the water content of the treated air and produce the humidified treated air. In some implementations, the humidifier 112 can include a compartment configured to house a sponge at least partially filled with water.

The anti-return valve 114 of the feed inlet 118 prevents backflow of the humidified treated air. The anti-return valve 114 can be configured to open when the user is breathing air into his lungs, and to close when the air is exhaled from the user's lungs. Various types of anti-return valves can be used for one-way flow of the air.

As seen in FIG. 1, the feed inlet 118 is coupled to the mask 116 that the user wears on her face such that the user can inhale the treated air following opening of the valve 114. The feed inlet 118 can be coupled to the mask 116 for instance via a tube or a conduit, the tube or conduit having a diameter suitable to ensure that a proper volume of humidified treated air can reach the user.

With reference to FIG. 7, in some implementations, the pre inhalation treatment section 22 can include a pressure regulator 120 coupled to the contaminant removal unit 104, an additional filter 122, such as a bacterial-viral filter, a feed inlet 124 comprising a valve 126, such as an anti-return valve, and a humidifier (not shown) coupled to the feed inlet 124 and receiving the treated air to produce a humidified treated air for supplying the humidified treated air to the mask 116. It is to be noted that the order of the various components included the pre inhalation treatment section 22 can vary. For instance, the humidifier can be provided upstream of the anti-return valve, etc. The characteristics of these components are similar as those described above.

With reference to FIG. 17, in some implementations, the pre inhalation treatment section 22 can include a flow control valve 128 coupled to the contaminant removal unit 104 to control the flow of treated air being delivered to the user, an additional filter 122, such as a bacterial-viral filter, and a feed inlet 124 comprising a valve 126, such as an anti-return valve. A humidifier can optionally be coupled to the feed inlet 124 and receive the treated air to produce a humidified treated air for supplying the humidified treated air to the mask 116.

### Pressurization of the Multistage Treatment System

As mentioned above, the pressure in the inhalation treatment unit 12 up to the pressure regulator can be set according to the pressure set point of the buffer tank 108. Such pressurization of the air intake section 14, the thermal treatment section 16, the UV treatment section 18 and the exhalation treatment unit 24, which can be a plasma reactor section, can be beneficial for the performance of the various treatment units. For instance, a higher pressure can improve the performance of the contaminant removal units, or traps. Also, when operating at a higher pressure, the flow velocity of the gas flowing through the various pathogen degradation units, for an equivalent volume at ambient pressure, is reduced. Reducing the flow velocity of the gas can increase the residence time of the gas to be filtered in the various pathogen degradation units, thereby potentially increasing their performance as well.

Moreover, the pressurisation of the system allows to fill in the buffer tank to provide a flow of treated air to the user at a pressure that is slightly above ambient pressure for ease of breath.

### Exhalation Treatment Unit

With reference to FIGS. 1, 7, 17A and 17B, the mask 116 can be further coupled to an exhalation treatment unit 24 for treating the exhaled air from the user. The exhaled air is supplied to the exhalation treatment unit 24 via an outlet line 130 coupled to the mask 116. Similarly to what is described above regarding the feed inlet 118 leading to the mask 116, the outlet line 130 comprises a valve 132, such as an anti-return valve, to prevent backflow of the exhaled air into the mask 116. In some implementations, the anti-return valve is configured to open when the user exhales.

The exhalation treatment unit 24 can include one or more pathogen degradation units. Suitable pathogen degradation units can include any one of the thermal treatment section, the UV treatment section, and the plasma reactor section as described above, or another type of pathogen degradation unit, provided in any suitable combination and order. In some implementations and as shown in FIGS. 1, 7 and 17, the exhalation treatment unit 24 includes a plasma reactor 134 integrated in an exhaust plasma reactor section 25.

### Exhaust Plasma Section

Still referring to FIGS. 1, 7, 17A and 17B, the exhalation treatment unit 24 includes an exhaust plasma reactor section 25 in fluid communication with the outlet line 130. The exhaust plasma reactor section 25 includes a plasma reactor 134 of similar configuration as the plasma reactor 134 of the inhalation treatment unit 12. The plasma reactor 134 includes an inlet, a plasma chamber comprising a gas flow path allowing a flow of the exhaled air to flow therethrough, a dielectric layer, a plasma generator and an outlet. The plasma generator is configured to apply a plasma-generating field across the flow of the exhaled air to generate a plasma therefrom, thereby producing a stream of treated exhaled air. In FIGS. 7, 17A and 17B, the plasma reactor 134 shown includes a solid conductive inner electrode and dialectic beads, but other configurations are also possible, such as those shown in FIG. 4.

In FIG. 1, the outlet is coupled to the plasma reactor 134 and receives the treated exhaled air, which is then flown through a valve 136, such as an anti-return valve, for expelling the treated exhaled air to the atmosphere. In some implementations and as illustrated in FIGS. 7, 17A and 17B, the valve 136 can be omitted and the treated exhaled air can be expelled to the atmosphere.

### Additional Characteristics of the Mask

In some implementations, the mask 116 coupled to the inhalation treatment unit 12 for receiving the treated air for inhalation by the user can include certain characteristics facilitating its proper fit and operation. The mask 116 includes a wall 138 defining an inhalation chamber between an inner surface and the face of the user. The pressure at which the treated air is provided to the inhalation chamber and the pressure in the inhalation chamber can be provided such that the inhalation chamber is pressurized, i.e., has a pressure above to ambient pressure. Pressurizing the inhalation chamber can contribute avoiding infiltration of air from the atmosphere via gaps that may be defined between the wall of the mask 116 and the face of the user, for instance if the mask 116 does not provide a proper fit.

### Additional Implementations of the Multistage Treatment System

In some implementations, the multistage treatment system can include various components of the treatment sections described above. Some of these combinations will now be described.

In some implementations, the multistage treatment system includes an inhalation treatment unit that includes a pressurized air intake section configured to receive and pressurize the potentially contaminated air, a plasma reactor section comprising a plasma chamber and a plasma generator configured to apply a plasma-generating field across the plasma chamber intersecting the flow of the pressurized air to generate a plasma therefrom, thereby producing a plasma treated stream that includes plasma-generated compounds. The multistage treatment system can further include a contaminant removal unit configured to receive the plasma treated stream and remove at least a portion of the plasma-generated compounds and produce treated air, and a feed inlet for supplying the treated air to a mask coupled such that the user can receive the treated air for inhalation by the user.

In some implementations, the multistage treatment system includes an inhalation treatment unit that includes a pressurized air intake section configured to receive potentially contaminated air. The pressurized air intake section includes an air pump to pressurize the particle-reduced stream and a check-valve allowing forward flow of the pressurized particle-reduced stream while preventing backflow. The inhalation treatment unit includes at least one pathogen degradation unit coupled to the pressurized air intake section and configured for destroying pathogens to produce treated air and byproducts. At least a portion of the byproducts can then be removed from the treated air via a byproduct removal unit coupled to the pathogen degradation unit. A feed inlet is provided to supply the treated air to the mask is from the byproduct removal unit so that the user can inhale the treated air.

In some implementations, the multistage treatment system includes an inhalation treatment unit that includes at least one treatment assembly configured to remove pathogens from the contaminated air to produce treated air. A feed inlet is provided to supply the treated air from the treatment assembly to the user. A mask is coupled to the feed inlet and receives the treated air for inhalation by the user. The multistage treatment system also includes an exhalation treatment unit coupled to the mask for treating exhaled air from the user. The exhalation treatment unit includes an exhaust plasma reactor section in fluid communication with the outlet line. The exhaust plasma reactor section includes an exhaust plasma chamber comprising a gas flow path allowing a flow of the exhaled air therethrough, and an exhaust plasma generator configured to apply a plasma-generating field across the exhaust plasma chamber intersecting the flow of the exhaled air to generate a plasma therefrom, thereby producing treated exhaled air. An outlet coupled to the exhaust plasma reactor section is provided to expel the treated exhaled air to the atmosphere.

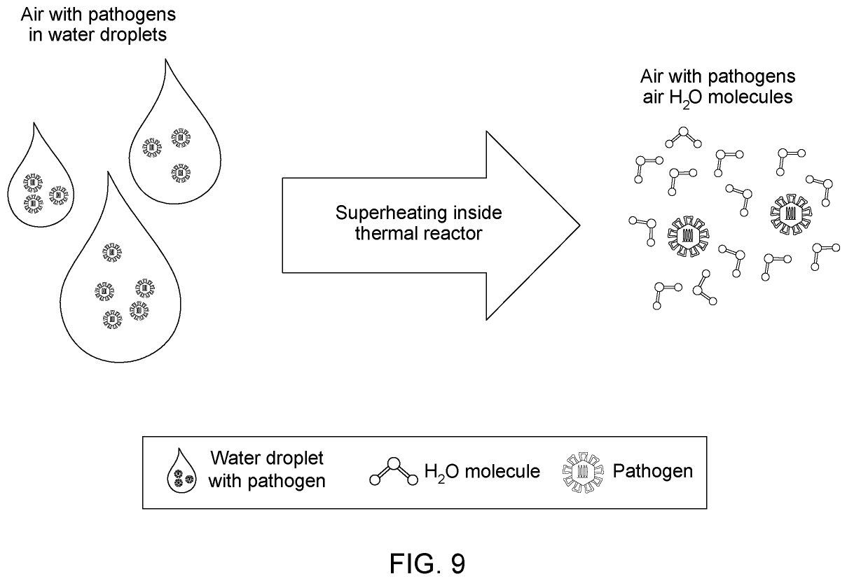

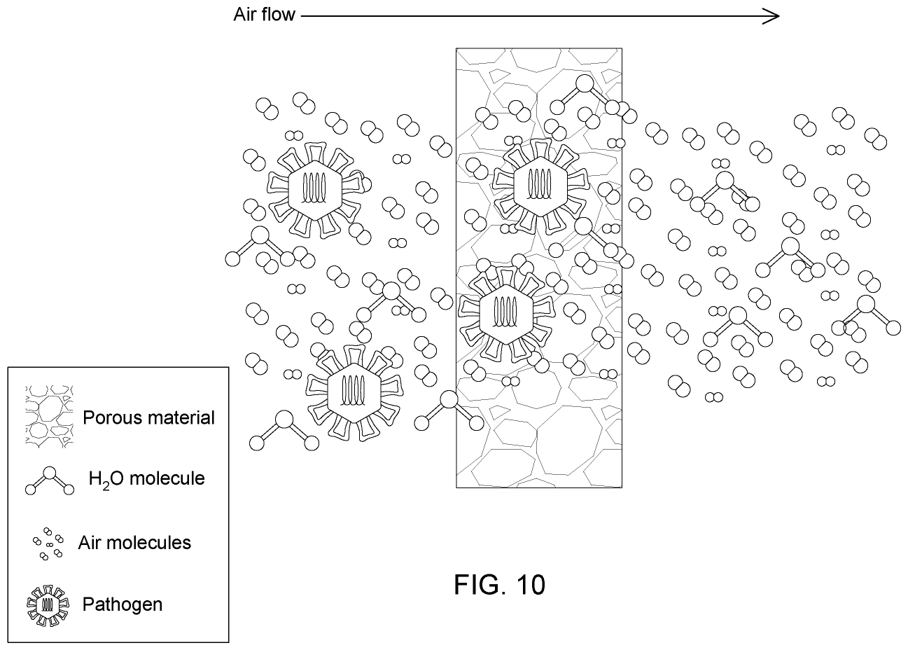

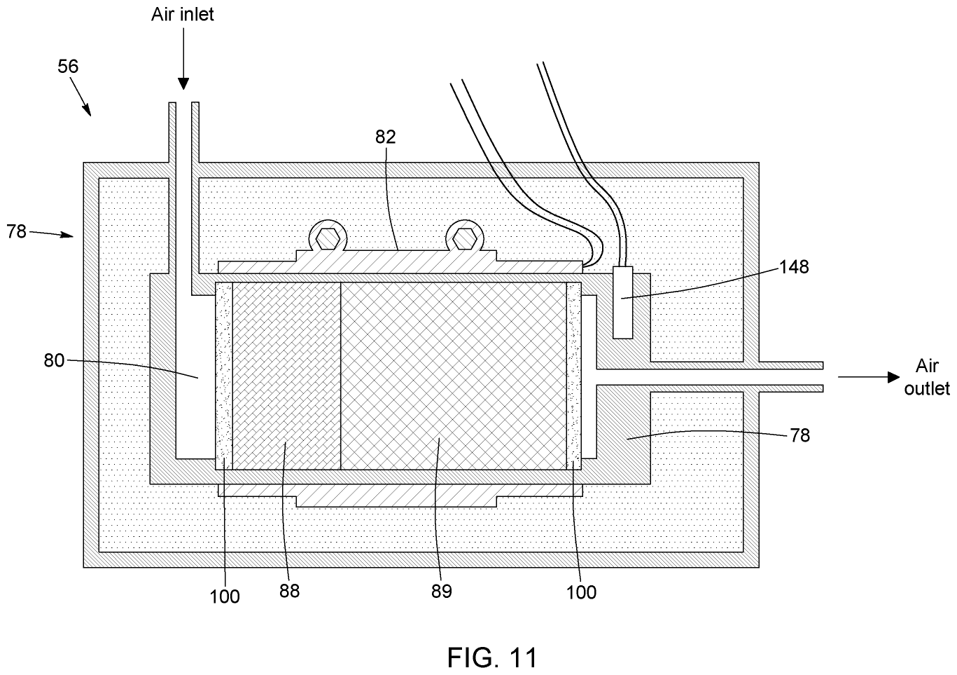

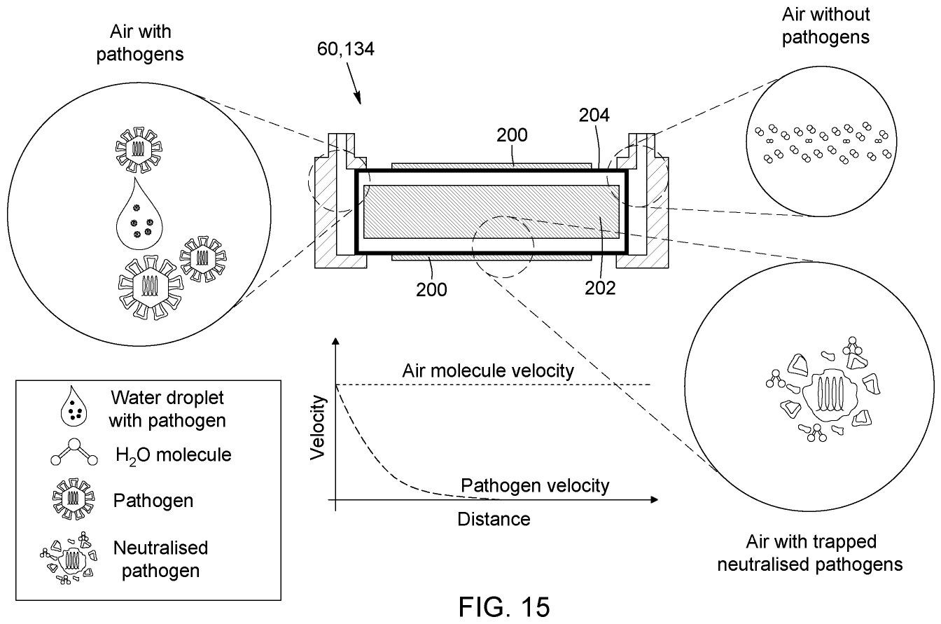

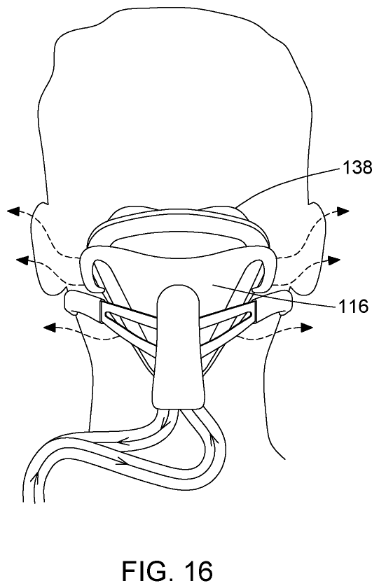

With reference now to FIGS. 7-15, another implementation of a multistage treatment system 10 will be described. The multistage treatment system includes an inhalation treatment unit 12 for treating air to be inhaled by the user. The inhalation treatment unit 12 includes an air intake section 14 configured to receive the contaminated air. The air intake section 14 can include a filter 50 configured to separate particles from the contaminated air and produce a particle-reduced stream, and an air pump 52 to pressurize the particle-reduced stream, or to increase pressure of the contaminated air. The filter 50 can be configured to separate coarse and/or fine particles, and can be adapted to the context in which the multistage treatment system. The air intake section 14 can also include an air distribution zone to accumulate a certain volume of air to be treated therein. In some implementations, the air pump 52 can be a blower that displaces air to be treated to the air distribution zone to facilitate an even distribution of the air to be treated in downstream sections of the multistage treatment system. A porous disk or a grid can also be included between the air distribution zone and the downstream sections of the multistage treatment system. In some implementations, the porous disk can have pores having a diameter ranging from 10 microns to 100 microns. In some implementations, the porous disk can have pores having a diameter larger than 100 microns.

Still referring to FIGS. 7-15, the inhalation treatment unit 12 further includes a thermal treatment section 16 in fluid communication with the air intake section 14. In the implementation shown in FIGS. 7 and 11, the thermal treatment section 16 includes a heating unit 56 configured to thermally treat the particle-reduced stream at a temperature sufficient to reduce a pathogen content thereof and produce a thermally treated stream, the heating unit 56 including a heating chamber 80, a first pathogen removal unit 88 and a second pathogen removal unit 89 received within the heating chamber 80, and optional porous disks 100 provided on either side of the first and second pathogen removal units 88, 89. The first pathogen removal unit 88 is configured to receive the particle-reduced stream and provide a first porous region 92 having an increased surface area for vaporizing water present in the contaminated air and remove vapour contaminants therefrom. The second pathogen removal unit 89 is configured to provide a second porous region 94 also having an increased surface area and vaporize water present in the contaminated air to further remove vapour contaminants therefrom. In some implementations, removing vapour contaminants can include retaining pathogens within one or both of the first and second porous region 92, 94. As mentioned above, the combination of heat provided to the heating unit 56 and the use of a pathogen removal unit can provide a means to dissociate pathogens from water droplets, to trap, or retain, the pathogens within the pathogen removal unit and to deactivate or destroy the trapped pathogens by the application of heat, and in some implementations, via various chemical reactions occurring within the pathogen removal units. The heating unit 56 further includes a cooling unit (not shown) to reduce the temperature of the thermally treated stream, and a contaminant removal unit 104 configured to receive the thermally treated stream and remove contaminants present initially in the air to be treated or generated during the thermal treatment. Examples of contaminants include VOCs, hydrocarbons, OH⁻, O₃, N₂O, CO, NOₓ and CO₂.

The treated air is then supplied to a pressure regulator 120 coupled to the contaminant removal unit 104, and passed through a bacteria filter 122 coupled to the pressure regulator 120. Optionally, a humidifier (not shown) coupled to the bacteria filter 122 can be present and configured to receive the treated air and producing a humidified treated air. A feed inlet 124 comprising an anti-return valve 126 then supplies the humidified treated air to a mask 116 coupled to the feed inlet for inhalation by the user.

Still referring to FIGS. 7-15, the multistage treatment system includes an exhalation treatment unit 24 coupled to the mask 116 for treating the exhaled air from the user. The exhalation treatment unit 24 includes an outlet line 130 coupled to the mask 116 and configured to receive the exhaled air from the user. The outlet line 130 can include an anti-return valve 140 to prevent backflow of the exhaled air into the mask. The exhaled air is then treated in an exhaust plasma reactor section 25 in fluid communication with the outlet line 130. The exhaust plasma reactor section 25 includes a plasma reactor 134 having an exhaust plasma chamber comprising a gas flow path allowing exhaled air to flow therethrough, and an exhaust plasma generator configured to apply a plasma-generating field across the exhaust plasma chamber intersecting the flow of the exhaled air to generate a plasma and produce treated exhaled air. An outlet coupled to the exhaust plasma reactor section and including an anti-return valve (not shown) then receives the treated exhaled air that will be expelled to the atmosphere.

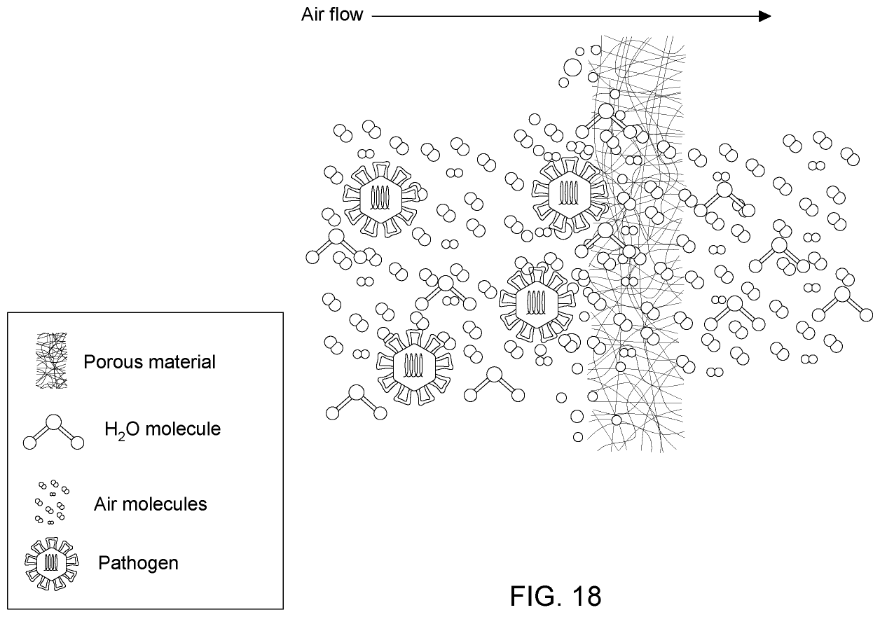

With reference now to FIGS. 17-20, another implementation of a multistage treatment system 10 will be described. The multistage treatment system includes an inhalation treatment unit 12 for treating air to be inhaled by the user. The inhalation treatment unit 12 includes an air intake section 14 configured to receive the contaminated air. The air intake section 14 can include a filter 50 configured to separate particles from the contaminated air and produce a particle-reduced stream, and an air pump 52 to pressurize the particle-reduced stream, or to increase pressure of the contaminated air. The filter 50 can be configured to separate coarse and/or fine particles, and can be adapted to the context in which the multistage treatment system. In some implementations, the air pump 52 can be a blower that displaces air to be treated to the air distribution zone to facilitate an even distribution of the air to be treated in downstream sections of the multistage treatment system.

In FIG. 17A, the air pump 52 is provided as part of the air intake section 14. Alternatively and with reference to FIG. 17B, the air pump 52 can be provided as part of the pre-inhalation treatment section 22, downstream of the bacterial filter 122, to suction in the air to be treated within the different sub-units of the inhalation treatment unit 12.

Still referring to FIGS. 17-20, the inhalation treatment unit 12 further includes a thermal treatment section 16 in fluid communication with the air intake section 14. In the implementation shown in FIGS. 17A, 17B and 19, the thermal treatment 16 section includes a heating unit 56 configured to thermally treat the particle-reduced stream at a temperature sufficient to deactivate pathogens contained in the air to be treated and produce a thermally treated stream. The heating unit 56 includes a heating chamber 80, a heating element 82, which can be a cartridge heater 84, configured to provide heat to the heating chamber 80, and a pathogen removal unit 88 received within the heating chamber 80. The pathogen removal unit 88 is configured to receive the particle-reduced stream and provide a porous region 92 configured to retain the pathogens therein, the porous region 92 being provided for instance by a porous material such as a metal filter, or metal mesh. The combination of the heat provided to the heating unit and the use of a pathogen removal unit 88 to retain the pathogens within the porous region 92 enables the retained pathogens to be exposed to a sufficiently high temperature and for a sufficiently long period of time, or residence time, to be deactivated while within the porous region 92. A heat exchanger can be provided upstream of the porous region 92 to enhance heat transfer between the air to be treated and the heat provided by the heating element 82 and optionally, to trap pathogens as well. A heating cycle can be implemented to alternate between periods of low temperatures and high temperatures within the heating chamber 80.

The heating unit 56 further includes a cooling unit (not shown) to reduce the temperature of the thermally treated stream, and a contaminant removal unit 104 configured to receive the thermally treated stream and remove contaminants present initially in the air to be treated or generated during the thermal treatment. Examples of contaminants include VOCs, hydrocarbons, OH⁻, O₃, N₂O, CO, NOₓ and CO₂.

The treated air is then supplied to a flow control valve 128 coupled with the contaminant removal unit 104, and passed through a bacteria filter 122 coupled to the pressure regulator 128. A feed inlet 124 comprising an anti-return valve 126 then supplies the humidified treated air to a mask 116 coupled to the feed inlet 124 for inhalation by the user.

Still referring to FIGS. 17-20, the multistage treatment system can include an exhalation treatment unit 24 coupled to the mask 116 for treating the exhaled air from the user. The exhalation treatment unit 24 includes an outlet line 130 coupled to the mask 116 and configured to receive the exhaled air from the user. The outlet line 130 can include an anti-return valve 140 to prevent backflow of the exhaled air into the mask. The exhaled air is then treated in an exhaust plasma reactor section 25 in fluid communication with the outlet line 130. The exhaust plasma reactor section 25 includes a plasma reactor 134 having an exhaust plasma chamber comprising a gas flow path allowing exhaled air to flow therethrough, and an exhaust plasma generator configured to apply a plasma-generating field across the exhaust plasma chamber intersecting the flow of the exhaled air to generate a plasma and produce treated exhaled air. An outlet coupled to the exhaust plasma reactor section and including an anti-return valve (not shown) then receives the treated exhaled air that will be expelled to the atmosphere.

### Control and Monitoring of the Multistage Treatment System

The multistage treatment system can include sensors to monitor various characteristics of the air stream as it flows through the components of the inhalation treatment unit 12 and/or the exhalation treatment unit 24. In some implementations, the sensors can be operatively connected to a corresponding controller to adjust a variable of the system in response to a measured value provided by the sensor. For instance, the thermal section 16 can include a temperature sensor and control system, the UV treatment section 18 can include a UV chamber control circuit and status monitoring, the plasma reactor section 20 can include a plasma reactor control circuit and status monitoring, and the pre-inhalation treatment section 22 can include a pressure monitoring and control circuit. An error management system and system status reporting for safety can also be provided.

Referring back to FIG. 1, the implementation shown includes a controller circuit that includes a temperature sensor 76 located in the thermal treatment section 16 and briefly described above, a pressure sensor 142 located in the pre-inhalation treatment section 22, a check valve state sensor 144 also provided in the pre-inhalation treatment 22, and a check valve state sensor 146 provided in the exhaust plasma section 25.

In this implementation, the temperature sensor 76 is configured to monitor the temperature of the heating unit 56. The heating unit 56 is operatively connected to a controller that controls the heat source providing heat to the heating unit 56. As mentioned above, when the temperature sensor 76 detects a temperature below a given lower threshold, the controller can turn on or heat up the heating unit, and when the temperature sensor detects a temperature above a given higher threshold, the controller can turn the heating unit off or down. In some implementations, the lower threshold can be between about 225° C. to about 275° C., while the higher threshold can be between about 325° C. and about 350° C. Controlling the heat provided to the heating unit 56 in such a way can contribute to reducing power consumption, which can contribute to prolonging battery life.

The pressure sensor 142 monitors a pressure in the buffer tank 108, which is also representative of the pressure in the inhalation treatment unit 12 upstream of the buffer tank 108. In some implementations, the multistage treatment system is operated at a high pressure to increase the performance of its various components with regard to the removal of contaminants. In addition, the pressure sensor 142 is operatively connected to the air pump, such that pressure in the system can be increased via the air pump 52 if the pressure is below a given pressure threshold. In addition, if the pressure sensor 142 detects that the system is below the given pressure threshold, an alarm will inform the user of a potential malfunction. Furthermore, the check valve state sensor 144 monitors the opening and closing configurations of the check valve 114, such that the pump 52 can be activated when the check valve 114 is in the closed configuration.

In some implementations, additional sensors can also be provided to detect a potential failure of the UV light source or of the plasma reactor, or to detect the battery status. In some implementations, an alarm will inform the user if either one of the heating unit, the UV unit and the plasma reactor encounters a malfunction. Advantageously, in implementations where the multistage treatment system includes more than one pathogen degradation unit, at least one pathogen degradation unit can remain operational if another one fails, thereby ensuring that the user can continue inhaling treated air.

FIG. 11 also illustrates a temperature sensor 148 operatively connected to the heating unit 56 to monitor the temperature at which the thermal treatment is conducted.

### Alternatives Implementations of the Inhalation Treatment Unit, the Exhalation Treatment Unit and the Mask Wearable on a User's Face

In some implementations, the inhalation treatment unit and/or the exhalation treatment unit can include, or consist essentially of, one or more filtration units, each filtration unit including a filter configured to perform a filtration stage and be subjected to a cleaning stage either sequentially or simultaneously, for a repetitive usage of the filter. The filter can be configured to retain, or trap, various airborne contaminants therein such as pathogens and particles, or any other types of contaminants that can be retained via filtration mechanisms. The filter can be made of a porous material, or porous media, defining a plurality of pores, or interstices. In turn, the porous material defines a tortuous path so that at least a majority of the contaminants collide with walls of the porous material, adhere thereto, and be retained within the porous material, i.e., within the filter. Depending on the porous material used, electrostatic attraction can also play a role in retaining the contaminants therein.

In some implementations, the inhalation treatment unit or the exhalation treatment unit can include a thermal treatment section comprising at least a heating unit provided upstream of the filtration unit. As previously described above, the heating unit can be configured to thermally treat air to be decontaminated at a temperature sufficient to reduce a pathogen content thereof and produce a thermally treated stream that is then supplied to the filtration unit. The heating unit can be any heating unit as described herein, for instance with reference to FIGS. 1, 7, 11, 12, 13, 17 and 19. In addition, the heating unit can include a butane burner or a propane burner, or any other type of fuel burner to provide heat to the heating unit. The heating unit provided upstream of the filtration unit can also be any type of heating unit that can be used to heat a stream of air to be decontaminated at a sufficient temperature and for a sufficient duration to deactivate at least a portion of pathogens in the contaminated air to be treated. In some implementations, a byproduct removal unit can be provided downstream of the heating unit to degrade or eliminate by products that may have been generated by the heating unit. The byproduct removal unit can include for instance at least one of a molecular sieve, a desiccant, and activated charcoal.

In some implementations, the inhalation treatment unit can include an air intake section that includes an inlet and an air pump, or blower. The blower is configured to drive the flow of air to be treated through the air intake section via the inlet and then through the filtration unit. The blower can also contribute to warm up, or heat, the air to be treated, which in turn can contribute to deactivate some pathogens at least in part. For instance, in some implementations, the blower can increase the temperature of the air to be treated by about 5° C. to about 10° C., by about 10° C. to about 15° C., or by about 15° C. to about 20° C., or up to about 30° C. For example, in one scenario, air to be treated can be at ambient temperature, e.g., at about 20° C., and the blower can heat up the air to be treated to a temperature of about 45° C.

In some implementations, when the inhalation treatment unit includes a heating unit and a blower, the blower can advantageously contribute to reduce the power supplied to the heating unit since the temperature of the air to be treated has already been raised by the action of the blower once it reaches the heating unit.

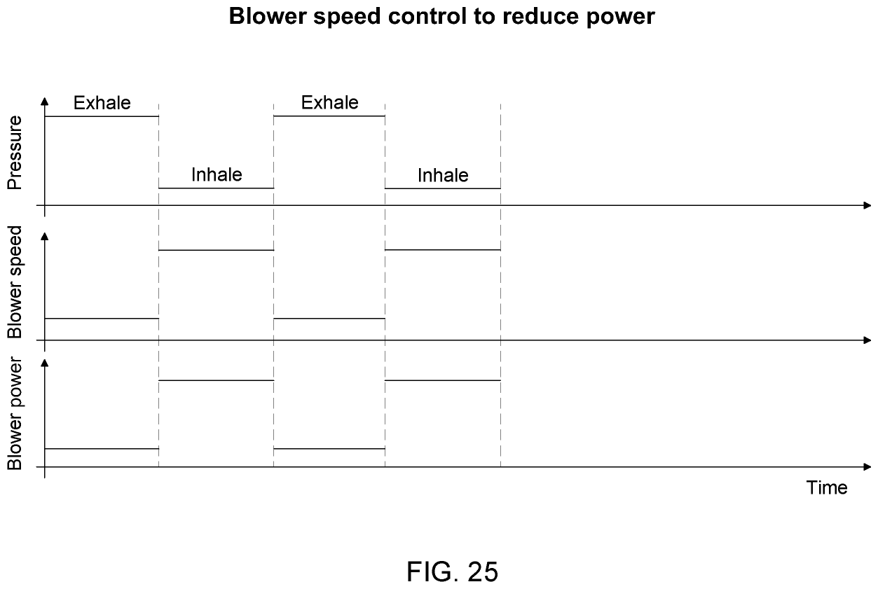

The mask coupled to the inhalation treatment unit for receiving the treated air for inhalation by the user includes a wall defining an inhalation chamber when the mask is installed on the face of the user. In some implementations, a pressure sensor can be provided to monitor a pressure within the inhalation chamber. The pressure sensor can be operatively connected to the blower, such that the pressure in the system can be increased via the air pump if the pressure is below a given pressure threshold. In addition, when the pressure sensor is operatively connected to the blower, the power of blower can be decreased when the pressure within the inhalation chamber is increased, for instance following exhalation by the user. This latter scenario is illustrated in FIG. 25, which shows three different timelines. The first timeline relates to a pressure in the inhalation chamber as a function of time. The second timeline relates to a blower speed in the inhalation chamber as a function of time.

The third timeline relates to a blower power in the inhalation chamber as a function of time. FIG. 25 illustrates that during a first period of time, i.e., during exhalation, the pressure within the inhalation chamber is higher than during a second period of time, i.e., during inhalation. The pressure within the inhalation chamber can thus vary in accordance with the breathing of the user. When the pressure sensor detects a high pressure in the inhalation chamber such as during exhalation, the blower speed and the blower power can be reduced, as shown in the second and third timelines, thereby advantageously decreasing the power consumption by the blower. The power consumption of the blower can thus advantageously be modulated in accordance with the pressure detected in the inhalation chamber.

In some implementations, the air intake section can include a valve configured to control the flow rate, or volume, of air to be treated supplied by the blower to the filtration unit and optionally to the heating unit if included in the inhalation treatment section. More particularly, the valve can be configured to at least partially close during exhalation by the user in order to reduce the volume of air to be treated supplied to the inhalation chamber during exhalation. In turn, reducing the volume of air to be treated supplied to the inhalation chamber during exhalation can advantageously contribute to reduce the power consumption by the blower. In some implementations, the blower can be in use during inhalation periods only, once again contributing to reduce the power consumption by the blower.

The porous material of the filter can be made of various materials. For instance, the porous material can be made of metal, fiberglass, polymer, ceramics such as alumina, or any other material that can be configured as a porous material that can collect contaminants therein and that can sustain the operating conditions at which it would be subjected to during the cleaning stage. Examples of metals can include stainless steel, or copper. In some implementations, the porous material made of metal, or metallic porous material, can be made of sintered metal fibers. In some implementations, the filter can include a coating that can contribute to deactivate pathogens. Examples of coatings can include various types of metal-organic frameworks, such as titanium based metal-organic frameworks. Metal organic frameworks have an inorganic-organic hybrid framework that comprise metal ions and organic ligands coordinated to the metal ions. Metal-organic frameworks can be designed to generate hydroxyl radicals, which in turn can contribute to damage organic material such as pathogens. AYRSORB™ T125 is an example of a metal-organic framework that can be used as a coating applied on the filter to contribute to deactivating pathogens retained therein.

The porous material can be chosen to achieve a given resistance to penetration of the contaminants within the porous material, so as to protect the wearer of the mask from these contaminants. The porous material can be characterized according to various parameters to determine its filtration capability. For instance, such parameters can include the most penetrating particle size (MPPS), which can be interpreted as corresponding to the size of particles that are most able to pass through the porous material. Another parameter can be the collection efficiency for particles having a given count median diameter (CMD) and a given mass median aerodynamic diameter. In some implementations, the porous material can be configured to retain contaminants having a mass median aerodynamic diameter of about 300 nm or less, between about 300 nm and about 100 nm, between about 100 nm and about 50 nm, between about 50 nm and about 10 nm, and between about 10 nm and 1 nm. In some implementations, the porous material can be configured to retain contaminants having a mass median aerodynamic diameter of less than about 5 nm, or less than about 2 nm. The porous material can also be configured to retain a certain percentage of contaminants having given physical characteristics such as a particle size diameter. For instance, in some implementations, the porous material can be configured to retain therein 95% of particles having a diameter larger than 1.5 nm. In some implementations, the porous material can be configured to retain therein 99% of particles having a diameter larger than 1.5 nm. In some implementations, the porous material can be configured to retain therein 99.99% of particles having a diameter larger than 1.5 nm. It is to be understood that when referring to contaminants being retained within the pores or interstices of the porous material, the contaminants can include pathogens and particles, or any other types of contaminants that can be retained via filtration mechanisms. Examples of particles can include those having a diameter that is less than 0.1 μm, or ultrafine particles.

Other properties of the filter and associated porous material such as its thickness, pore size and packing density can also be used to characterize the porous material. In some implementations, the pore size of the porous material can range from between about 20 nm to about 5 nm, or from between about 15 nm to about 5 nm. In some implementations, the pore size of the porous material can be approximately 10 nm. In some implementations, the thickness of the filter can range from about 1 cm to about 5 mm. The pore size or the packing density of the filter can also be chosen such that the pressure drop or resistance to airflow across the filter is maintained within a certain range that enables proper breathing by the user.

The physical characteristics of the filter can be modified and adapted in accordance with the intended application. Examples of physical characteristics of the filter that can be varied include the choice of porous material, the configuration of the porous material in terms of pore size and packing density, the thickness of the filter, and the superficy of the filter.

As mentioned above, the filter can be configured to perform a filtration stage and to be subjected to a cleaning stage, either sequentially or simultaneously, to enable a repetitive usage of the filter. In some implementations, the filter can be removed by the user from the inhalation treatment unit or the exhalation treatment unit to be cleaned, e.g., according to decontamination and sterilization techniques, and once cleaned, the filter can then be repositioned to its operational location by the user and be re-used for filtration. The filter can thus be removed from its operational location, i.e., the location where the filtration unit is installed to fulfill its function of filtration, to be cleaned and decontaminated at a different location. For instance, a user can remove the filter from the inhalation treatment unit or the exhalation treatment unit, and subject the filter to a cleaning stage at a different location where suitable equipment is available to perform the cleaning stage. When the filtering unit is integrated into the mask, the user can perform substantially the same steps by removing the filter from the filtration unit and subject the filter to a cleaning stage at a different location where suitable equipment is available to perform the cleaning stage. According to this scenario, the filtration stage and the cleaning stage would be considered as being performed sequentially. In other implementations, the filter can remain in its operational location, and the cleaning stage can be performed as the filtering unit is in operation, i.e., while the mask is in use. According to this scenario, the filtration stage and the cleaning stage would be considered as being performed simultaneously. In yet other implementations, the filter can remain at its operational location within the filtration unit, although the mask may be removed from the user's face following a filtration stage to be subjected to the cleaning stage. This scenario can occur for instance when compounds that may not be suitable for inhalation by the user are generated during the cleaning stage, or simply for convenience purposes. According to this scenario, the filtration stage and the cleaning stage would be considered as being performed sequentially.

The cleaning stage is performed to deactivate and remove contaminants that have been trapped in the porous material during usage. Various options are possible with regard to the cleaning stage, some of which being described below.

Thermal Treatment

The cleaning stage can include subjecting the filter to a thermal treatment at a given temperature known to at least deactivate pathogens such as viruses and bacteria, for a given duration. In some implementations, operating conditions of the thermal treatment can be such that the pathogens can be destroyed.

The thermal treatment can be operated at various conditions. The conditions can vary depending for instance whether a wet thermal treatment or a dry thermal treatment is to be performed.

In some implementations, a wet thermal treatment for a filter having a porous material made of metal can include submerging the filter in an aqueous medium, such as water, and boiling the aqueous medium for a period of time that can range from 1 minute to 5 minutes, or any other duration suitable for decontamination of the filter.

In some implementations, a dry thermal treatment can include heating the filter to at a temperature of about 65° C. for a duration between about 5 to about 30 minutes, or any other duration suitable for decontamination of the filter.

Different combinations of temperatures and duration can be suitable, in accordance with the general concept that for a lower temperature of the thermal treatment, the duration of the thermal treatment is generally longer. Different operating parameters of the thermal treatment can be also determined to be suitable in accordance with the types of contaminants that are retained in the porous material of the filter.

In some implementations, depending on the material from which is made the porous material, the cleaning stage can include subjecting the filter to an autoclave treatment, which can sterilize the filter.

In some implementations, when the cleaning stage includes a thermal treatment, the cleaning stage can be performed following the removal of the filter from its operational location. Alternatively, the filter can remain at its operational location within the filtration unit when subjected to the thermal treatment, for instance following removal of the mask from the user's face.

Microwave Treatment

In some implementations, depending on the material from which the porous material is made, the cleaning stage can include subjecting the filter to microwaves for a duration sufficient to deactivate pathogens. For example, a porous material made of a polymer can sustain a cleaning stage performed in a microwave.

Application of an Electric Field

In some implementations, when the filter is made of a metallic porous material, the cleaning stage can include applying an electric current through the filter. The electric current can be an alternating current (AC) or a direct current (DC). The application of an electric current through the filter generates an electric field that can have a biocidal effect and deactivate the pathogens that have been retained in the filter.

A static electric field can also be applied to deactivate pathogens. The application of a static electric field can also facilitate removing particles, similarly to an electrostatic precipitator.

Of note, in this implementation of the cleaning stage, the cleaning stage can be performed following the removal of the filter from its operational location. Alternatively, the cleaning stage can also be performed while the filter remains at its operational location. Thus, when the application of an electric field is used for the cleaning stage, the filtration stage and the cleaning stage can be performed either sequentially or simultaneously. In other words, the electric current can be applied during the filtration stage to deactivate the pathogens as they are being trapped in the porous material, thereby performing the cleaning stage at the same time as the filtration stage as the mask is being worn by the user, which would correspond to the filtration stage and the cleaning stage being performed simultaneously. Alternatively, the mask can be removed from the user's face, and an electric current can be applied through the filter for a given duration to perform the cleaning stage, and then be ready to be worn again by the user. The filter can also be removed from its operational location, and an electric current can be applied through the filter for a given duration to perform the cleaning stage, and the cleaned filter can be placed back into its operational location and be ready to be worn again by the user. These two scenarios would correspond to the filtration stage and the cleaning stage being performed sequentially.

UV Treatment

In some implementations, the cleaning stage can include subjecting the filter to a UV treatment. In order to do so, the filter can be removed from the filtration unit and be exposed to UV radiations according to techniques known in the art. In this implementation, the filter can be removed from its operational location to be subjected to the cleaning stage. This scenario would correspond to the filtration stage and the cleaning stage being performed sequentially. Alternatively, the filtration unit can include a UV light source configured to emit UV radiation, and be configured to define a UV chamber into which the filter can be received. During the cleaning stage, the UV light source can emit UV radiation to kill or deactivate pathogens retained in the filter, with the filter remaining at its operational location. In such implementations, the filtration stage and the cleaning stage can be performed simultaneously or sequentially.

Plasma Treatment

In yet other implementations, the cleaning stage can include subjecting the filter to a plasma treatment. In such implementations, the filtration unit can include a plasma generator configured to apply a plasma-generating field across the flow of air passing through the filter to generate a plasma therefrom, thereby deactivating pathogens retain within the porous material and producing a plasma treated stream. Since the generation of a plasma can also produce plasma-generated compounds, such as N₂O, NOx, and/or ozone, this type of cleaning stage can be performed while the filter has been removed from its operational location, which would correspond to the filtration stage and the cleaning stage being performed sequentially, or while remaining at its operational location. If the plasma treatment is to be conducted while the filter remains at its operational location, the mask would preferably not be in use given the potential production of plasma-generated compounds which may not be suitable for inhalation, which would still correspond to the filtration stage and the cleaning stage being performed sequentially. If the mask is to remain in use during the plasma treatment, an additional filter configured to capture plasma-generated compounds can be coupled to the filter made of a porous material, in which case the filtration stage and the cleaning stage could be performed simultaneously.

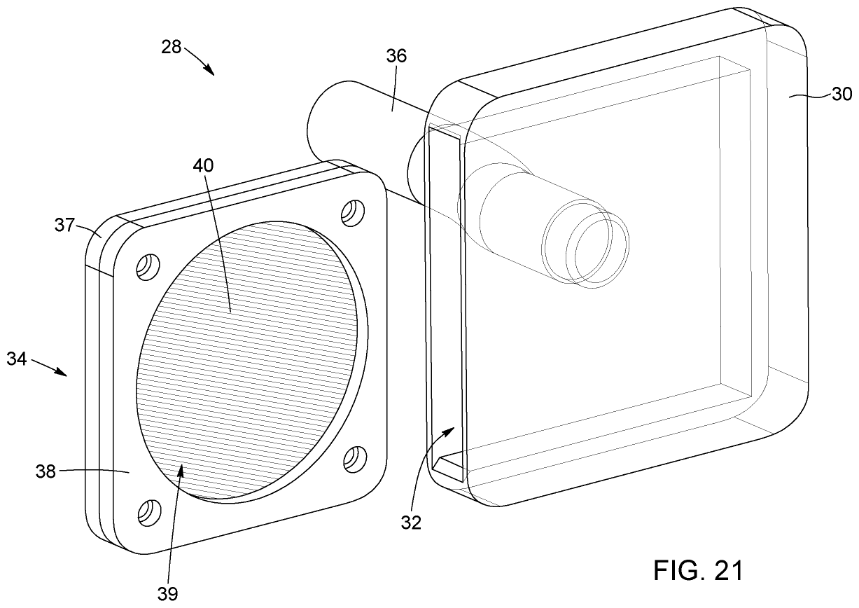

With reference now to FIGS. 21 to 23, examples of implementations of a filtration unit will now be described in further detail.

FIG. 21 illustrates a filtration unit 28 comprising a casing 30 defining a cartridge-receiving section 32. The filtration unit 28 also includes a cartridge 34 and a conduit 36, the conduit 36 being configured to establish fluid communication between the mask and the filtration unit. In the illustrated implementations, the cartridge 34 includes a filter-retaining frame 38, and a filter 40 as discussed above. The cartridge 34 is insertable into the cartridge-receiving section 32 and once inserted therein, can remain in place for the duration of the filtration stage. Following the filtration stage, the cartridge 34 can be removed from the cartridge-receiving section 32, and be subjected to the cleaning stage. It is to be noted that the cartridge 34 in its entirety can be subjected to the cleaning stage, or alternatively, the filter 40 can be uncoupled from the filter-retaining frame 38 to be subjected to the cleaning stage. The cartridge 34 can be re-used for several cycles of filtration stage and cleaning stage. In some implementations, the cartridge 34 can be re-used indefinitely as long as the physical integrity of the porous material remains suitable to perform the intended filtration purpose of the filter 40. In some implementations, the cartridge 34 can also be replaced by a new cartridge following a certain number of cycles, or if it is suspected that the physical integrity of the porous material has been compromised. The interaction between the cartridge 34 and the cartridge-receiving section 32 of the casing 30 is such that passage of air other than through the filter 40 is minimized once the cartridge 34 is inserted into the cartridge-receiving section 32. In some implementations, interaction between the cartridge 34 and the cartridge-receiving section 32 once the cartridge 34 is inserted into the cartridge-receiving section 32 can result in an air-tight seal, with air flowing solely though the porous material of the filter 40.

In the implementation shown in FIG. 21, the cartridge-receiving section 32 is connected to a flow conduit 36, or tube, that extends to the inside of the mask to deliver air that has been filtered by the filter to the used. In this implementation, the filtration unit 28 can form part of an inhalation treatment unit, or the inhalation treatment unit can consist essentially of the filtration unit. Similarly, the filtration unit 28 can form part of an exhalation treatment unit, or the exhalation treatment unit can consist essentially of the filtration unit.

As mentioned above, the cartridge 34 includes a filter-retaining frame 38 and a filter 40. The filter-retaining frame 38 can be any type of structure that enables retaining the filter 40 in place. In the implementation shown, the filter-retaining frame 38 includes two substantially rectangular frame portions 37 that are configured to sandwich the filter 40 therebetween, each of the frame portions 37 defining an opening 39 enabling the filter 40 to be exposed on both sides thereof. It is to be understood that the implementation of the filter-retaining frame 38 shown in FIG. 21 is for illustrative purposes only, and that various other shapes and configurations are also suitable.

In addition, the filter 40 can include more than one layer of porous material. For instance, a first layer of porous material, i.e., the layer that would be contacted with the air to be decontaminated first, can be made of a porous material having certain properties, while a second layer of porous material can be provided adjacent and downstream to the first layer of porous material, the second layer of porous material having also properties that may be similar or different from the properties of the first layer of porous material. Providing a first and second layers of porous material can thus result in a multi-layer porous material. The first and second porous materials can be made for instance of different materials, or have a different pore size. More than two layers of porous material can also be provided, and would also result in a multi-layer porous material.

It is also to be noted that in some scenarios, the cartridge-retaining frame 38 can be omitted, and the filter 40 can be configured to be inserted as a standalone component into the cartridge-receiving section 32 which, in this scenario, could accordingly be referred to as a filter-receiving portion.

It is also to be noted that although the combination of the cartridge-receiving section 32 and the cartridge 34 are shown in FIG. 21 as being coupled via an insertion mechanism that includes a lateral sliding motion of the cartridge 34 into the cartridge-receiving section 32, other types of interaction between the cartridge-receiving section 32 and the cartridge 34 are also possible. For instance, the cartridge can be combined with the cartridge holder, or casing 30, via a snap-on mechanism.

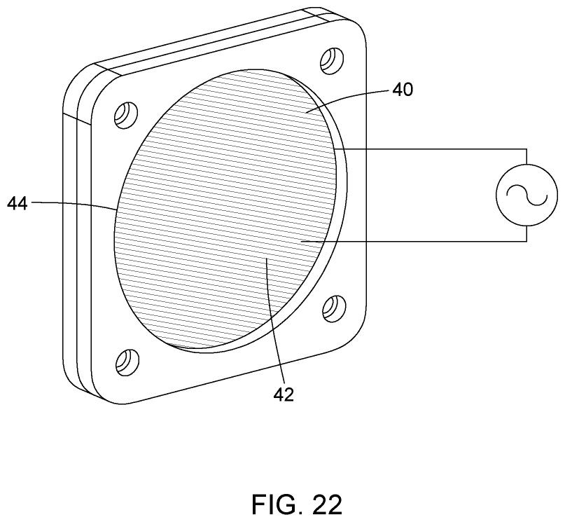

With reference to FIG. 22, in implementations where the cleaning stage includes applying an electric field through a metallic porous material, the filter 40 can act an electrode 42, and the cartridge 34 can further include an electrically insulated gasket 44. The electrically insulated gasket 44 can prevent electric current to propagate further out then the filter 40, for safety reasons. FIG. 22 further illustrates the DC or AC high voltage source that enables applying the electric current across the filter 40. In such implementations, the filter 40 can form a first electrode, and the filter-retaining frame 38 can form a second electrode, with the electric current applied therebetween.

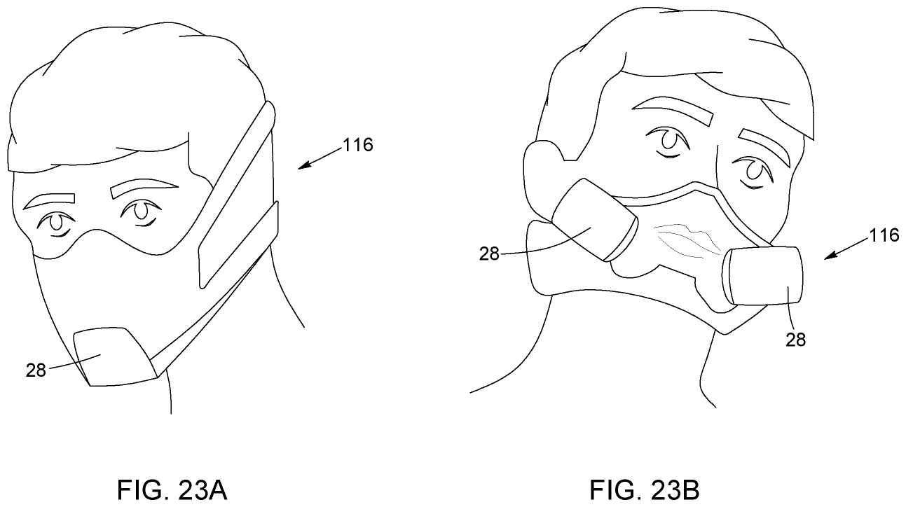

With reference now to FIGS. 23A and 23B, in other implementations, the filtration unit 28 can be integrated into the mask 116 as its operation location instead of being provided separately from the mask 116 but in fluid communication therewith, such as when forming part of the inhalation treatment unit and/or the exhalation treatment unit. In implementations where the filtration unit 28 is provided as being integrated into the mask 116, the filtration unit 28 can include a single filter, or two or more filters can be provided. When two or more filters are provided, they can be provided in series, so as to form a succession of filters through which the flow of air can pass. The succession of filters can be provided in an adjacent relationship, the filters can be spaced apart from one another. In some implementations, more two or more filtration units 28 can also be provided and integrated into the mask 116. For instance, FIG. 23B illustrates an implementation wherein two filtration units are integrated into the mask.

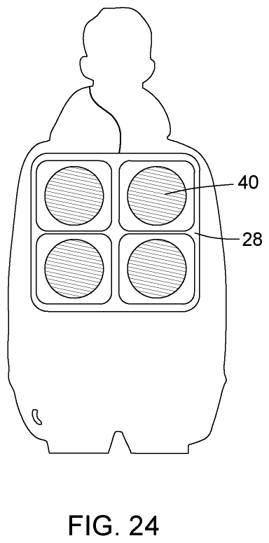

In some implementations, a filtration unit 28 can include two or more filters 40 that are provided in a side-by-side relationship, such as shown in FIG. 24. In such implementations, the filters 40 can also be said to be provided parallelly to each other, or within a same plan that is somewhat parallel to the frontal plane of the body of the user. Providing filters in a side-by-side relationship can facilitate reducing the restriction to airflow through the filter while providing an increased surface area of filtration compared to when a single filter would be used. Providing filters in a side-by-side relationship can thus facilitate breathing since restriction to airflow is reduced. This type of configuration of the filters can be used for instance when no air pump, or blower, is used to force the airflow through the filters, since restriction to airflow is already reduced by the side-by-side relationship of the filters. Although FIG. 24 illustrates four filters 40 provided in a side-by-side relationship as part of a filtration unit 28 that is worn on the back of the user, it is to be understood that filters can be provided in a side-by-side relationship when the filtration unit is integrated into the mask. Filters can also be provided in a side-by-side relationship when the filtration unit forms part of an inhalation treatment unit or an exhalation treatment unit, or when the inhalation treatment unit or the exhalation treatment unit consists essentially of the filtration unit.

Different techniques can be used to integrate the one or more filtration units to form part of the mask. For instance, the mask can be made of a polymer, e.g., silicone or polyethylene, and can include a filtering unit-receiving opening, or window, to receive the cartridge-receiving section. The cartridge-receiving section can then be bonded, glued or sealed to the outer perimeter of the opening. Any other type of suitable technique can be used to integrate the filtering unit to the mask such that once positioned on the user's face, the combination of the mask and the filtration unit facilitate preventing infiltration of contaminated air at locations other than through the filter of the filtration unit.

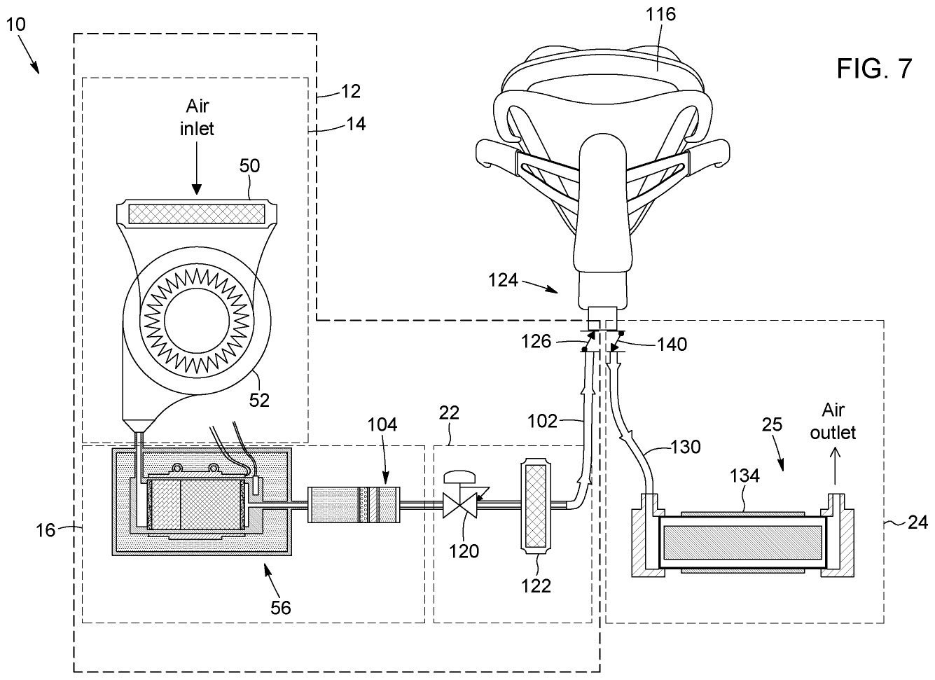

As mentioned above, the filtration unit 28 can be included in an inhalation treatment unit and/or an exhalation treatment unit as described herein. In some implementations, the inhalation treatment unit and/or the exhalation treatment unit in which the filtration unit 28 is received can be provided separately from the mask but in fluid communication therewith, and the inhalation treatment unit and/or the exhalation treatment unit can be worn for instance at the belt of the user or in the back of the user, i.e., as a backpack. FIG. 6 illustrates an example of an inhalation treatment unit 12 and/or exhalation treatment unit that is configured to be worn at the belt, with a filtration unit that can be received therein.

In some implementations, when the filtration unit 28 is integrated into the mask, the conduit 36 previously described with reference to FIG. 21 can be omitted.

Furthermore, in implementations where the filtration unit 28 is integrated into the mask, the filtering unit 28 can advantageously be operational to filter contaminants in a bi-directional fashion. In other words, contaminated air that includes airborne contaminants can be treated as it passes from the environment through the filter 40 of the filtering unit 28 in an inflow direction to produce filtered air that can be delivered to the user for inhalation. In turn, contaminated air resulting from exhalation by the user, which also may contain contaminants such as pathogens, can be treated as it passes through the filter 40 of the filtration unit 28, in an outflow direction, prior to being released to the atmosphere.

### Additional Applications of the Multistage Treatment System

In some implementations, selected components, or sub-units, of the multistage treatment system can be omitted depending on the planned usage. For instance, in some implementations, the multistage treatment system can include at least an inhalation treatment unit and a pre inhalation treatment section, i.e., with or without an exhalation treatment unit. The multistage treatment unit can be configured to be used for any application that requires a provision of treated air, for instance to fill up a compressed air cylinder or gas tank. An application for this configuration of the multistage treatment unit can be for example for apparatus that require a treated air supply, such as a culture chamber, a bioreactor, an incubator, or the like. This configuration of the multistage treatment unit can also be useful for providing treated air to a user also via a mask, but without the need for treating the exhaled air, for instance in the case of firefighters.

Furthermore, the multistage treatment system can be scaled to treat a desired volume of gas. The multistage treatment system can be scaled so as to provide a sufficient volume of treated air for usage by a single user, or the multistage treatment system can be scaled for laboratory applications, or for industrial applications that require a large volume of treated gas.

Several alternative implementations and examples have been described and illustrated herein. The implementations of the technology described above are intended to be exemplary only. A person of ordinary skill in the art would appreciate the features of the individual implementations, and the possible combinations and variations of the components. A person of ordinary skill in the art would further appreciate that any of the implementations could be provided in any combination with the other implementations disclosed herein. It is understood that the technology may be embodied in other specific forms without departing from the central characteristics thereof. The present examples and implementations, therefore, are to be considered in all respects as illustrative and not restrictive, and the technology is not to be limited to the details given herein. Accordingly, while the specific implementations have been illustrated and described, numerous modifications come to mind.

1-260. (canceled)

261. A device for treating contaminated air for inhalation by a user, the device comprising:
an inhalation treatment unit for treating air to be inhaled by the user, comprising:
an air intake section configured to receive the contaminated air;
a thermal treatment section in fluid communication with the air intake section, the thermal treatment section comprising:
a heating unit configured to thermally treat the contaminated air at a temperature sufficient to deactivate pathogens contained therein and produce a thermally treated stream, the heating unit comprising:
a heating chamber;
a heating element configured to provide heat to the heating chamber;
a pathogen removal unit received within the heating chamber, the pathogen removal unit being configured to provide a porous region to retain the pathogens therein;
a contaminant removal unit configured to receive the thermally treated stream and remove byproducts from the thermal treatment section and produce treated air;
a feed inlet comprising an anti-return valve for supplying the treated air;
a mask coupled to the feed inlet for receiving the treated air for inhalation by the user.

262. The device of claim 261, wherein the porous region comprises a metal mesh.

263. The device of claim 261, wherein the porous region has a pore size between about 1 nm and 10 nm.

264. The device of claim 261, wherein the porous region has a pore size of less than about 1 nm.

265. The device of claim 261, wherein the pathogen removal unit is configured to increase a residence time of pathogens within the heating chamber.

266. The device of claim 261, wherein the air intake section comprises a filter configured to separate particles from the contaminated air, and an air pump to pressurize the contaminated air.

267. The device of claim 261, further comprising an exhalation treatment unit coupled to the mask for treating the exhaled air from the user.

268. The device of claim 267, wherein the exhalation treatment unit comprises:
an outlet line coupled to the mask and configured to receive the exhaled air from the user, the outlet line comprising an anti-return valve; and
an exhaust plasma reactor section in fluid communication with the outlet line.

269. The device of claim 267, wherein the exhaust plasma reactor section comprises:
an exhaust plasma chamber comprising a gas flow path allowing a flow of the exhaled air therethrough; and
an exhaust plasma generator configured to apply a plasma-generating field across the exhaust plasma chamber intersecting the flow of the exhaled air to generate a plasma therefrom, thereby producing treated exhaled air.

270. The device of claim 269, further comprising an outlet coupled to the exhaust plasma reactor section for receiving the treated exhaled air and comprising an anti-return valve for expelling the treated exhaled air to the atmosphere.

271. A device for treating contaminated air for inhalation by a user, the device comprising:
a thermal treatment section comprising:
a heating unit configured to thermally treat the contaminated air at a temperature sufficient to reduce a pathogen content thereof and produce treated air, the heating unit comprising:
at least one pathogen removal unit configured to receive the contaminated air and provide a porous region for exposing pathogens to heat;
a feed inlet for supplying the treated air; and
a mask coupled to the feed inlet for receiving the treated air for inhalation by the user.

272. The device of claim 271, wherein the heating unit comprises a heating chamber, and the at least one pathogen removal unit is received within the heating chamber.

273. The device of claim 272, wherein the heating unit further comprises a heat exchanger configured to be received within the heating chamber.

274. The device of claim 273, wherein the heat exchanger is provided upstream of the porous region.

275. The device of claim 274, wherein the heat exchanger is configured to provide an additional porous region.

276. The device of claim 275, wherein the additional porous region has larger pores than the porous region.

277. The device of claim 273, wherein the heat exchanger comprises a metal wool.

278. The device of claim 271, wherein the heating unit comprises a heater cartridge.

279. The device of claim 272, wherein the pathogen removal unit is configured to increase a residence time of the pathogens within the heating chamber.

280. The device of claim 271, wherein the porous region has a pore size between about 1 nm and 10 nm.

281. The device of claim 271, wherein the porous region has a pore size of less than about 1 nm.

282. The device of claim 271, wherein the porous region comprises a metal mesh.

283. The device of claim 282, wherein the metal mesh comprises sintered metal fibers.

284. The device of claim 282, wherein the metal mesh comprises multiple layers of sintered metal fibers to form a multi-layer metal mesh.

285. The device of claim 283, wherein the sintered metal fibers are configured to lay substantially uniformly to form a three-dimensional non-woven structure.

286. The device of claim 284, wherein the pore size of at least one layer of the multi-layer metal mesh is different than the pore size of the remaining layers.

287. The device of claim 271, further comprising a temperature sensor to monitor the temperature within the heating chamber and a controller operatively connected to the temperature sensor and to the heating element, the controller being configured to adjust the temperature within the thermal unit in response to a measured temperature value provided by the temperature sensor.

288. The device of claim 287, wherein the controller is configured to adjust the temperature within the thermal unit according to a heating cycle.

289. The device of claim 288, wherein the heating cycle comprises a temperature sequence comprising a low temperature setpoint and a high temperature setpoint.
